(19) United States
(12) Patent Application Publication
(10) Pub. No.: US 2025/0010278 A1
(43) Pub. Date: Jan. 9, 2025

(54) STERICALLY MODIFIED SCHIFF BASE LIGANDS FOR ENHANCED CATALYTIC CARBONYLATION ACTIVITY

(71) Applicant: Novomer, Inc., Rochester, NY (US)

(72) Inventors: Jonathan D. Tedder, Rochester, NY (US); Geoffrey W. Coates, Lansing, NY (US); Catherine A. Falkner, Rochester, NY (US); Lisa B. Todd, Rochester, NY (US); Ronald Boyce, Rochester, NY (US); Jocelyn Shyong, Rochester, NY (US)

(21) Appl. No.: 18/576,970

(22) PCT Filed: Jul. 7, 2022
(86) PCT No.: PCT/US2022/073501

§ 371 Date: Jan. 5, 2024

Related U.S. Application Data
(60) Provisional application No. 63/220,126, filed on Jul. 9, 2021.

(51) Int. Cl. B01J 31/22; B01J 31/20; C07D 305/12

(52) U.S. Cl. CPC B01J 31/2243; B01J 31/20; C07D 305/12; B01J 2231/34; B01J 2531/0252; B01J 2531/31

(57) ABSTRACT

A composition, comprising: a metal carbonyl anion; and a cation ionically bonded to the metal carbonyl anion. The cation includes a ligand and a metal centered compound. The ligand includes two residues of 3,5-substituted salicylaldehydes connected by an hydrocarbyl-diimine bridge that includes a nitrogen atom contacted with a carbon of an aldehyde residue at each of the two residues of the 3,5-substituted salicylaldehydes. Each of the residues of the 3,5-substituted salicylaldehydes are independently substituted at one or both of a 3 position and a 5 position by a hydrocarbyl group containing at least 5 carbons. The metal coordinated with the ligand at each hydroxyl residue the two residues of the 3,5-substituted salicylaldehydes at a 2 position and at each of the nitrogen atoms of the hydrocarbyl-diimine bridge. The composition includes two polar ligands coordinated with the metal.

### FIELD

The present disclosure relates to novel sterically modified Schiff base ligands in catalysts used in the production of lactones from epoxides and relates to methods of making the catalyst and using the catalyst to make lactones.

### BACKGROUND

Carbonylation is a process that can be used to react carbon monoxide and an epoxide to make a lactone. In some cases, additional steps are taken to react the lactones to make polymers. These lactones or polymers thereof are often used as plastics and disinfectants. When making these lactones, a carbonylation catalyst is used to optimize the efficiency of the reaction to produce lactones at competitive prices. Carbonylation catalysts are expensive, and thus, new carbonylation catalysts and new techniques to synthesize the carbonylation catalysts from simple components are needed. Some catalysts have been made using Schiff based ligands. For example, see U.S. Pat. No. 6,852,865. However, these catalysts have not been shown to produce high enough yields of lactones to make the catalyst practicable.

Accordingly, what is needed are catalysts used in carbonylation processes to make lactones that can be easily assembled and have high efficiency in production of lactones.

### SUMMARY

Disclosed are compositions including a metal carbonyl anion and a cation ionically bonded to the metal carbonyl anion. The cation includes a ligand having two residues of 3,5-substituted salicylaldehydes connected by a hydrocarbyl-diimine bridge that includes a nitrogen atom contacted with a carbon of an aldehyde residue at each of the two residues of the 3,5-substituted salicylaldehydes. Each of the residues of the 3,5-substituted salicylaldehydes are independently substituted at one or both of a 3 position and a 5 position by a hydrocarbyl group containing at least 5 carbons. The cation includes a metal coordinated with the ligand at each hydroxyl residue and the two residues of the 3,5-substituted salicylaldehydes at a 2 position and at each of the nitrogen atoms of the hydrocarbyl-diimine bridge. The cation includes two polar ligands coordinated with the metal.

Disclosed herein is a method of making the composition including contacting a ligand with a metalating agent to form a metal centered compound. The ligand includes the residues of two 3, 5-substituted salicylaldehydes connected by a hydrocarbyl-diimine bridge at the aldehyde residue of the two residues of the 3,5-substituted salicylaldehydes. One or both of a 3 position and a 5 position of each of the two residues of the 3,5-substituted salicylaldehydes is a hydrocarbyl group containing at least 5 carbons. The method further includes contacting the metal centered compound with a metal carbonyl and a polar ligand to form a composition.

Disclosed herein is a method of making beta propiolactone from the composition by contacting an epoxide and carbon monoxide in the presence of a composition that is a carbonylation catalyst to form beta propiolactone.

The composition may have the following structure:

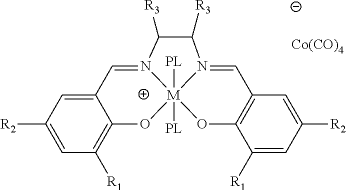

wherein M is a metal;
wherein PL is a polar ligand;
   wherein each R₁ is independently selected from one or more of an alkyl group, an aryl group, or an alkyl-aryl group;
   wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
   wherein at least one of R₁ or R₂ is selected from an alkyl group, an aryl group, or an alkyl-aryl group that contains at least 5 carbons; and
   wherein each R₃ is independently selected from hydrogen, methyl, a C₂₋₁₀ alkyl group, a combination thereof, or in combination form a six membered ring that is optionally a substituted aromatic ring.

The ligand may have the following structure:

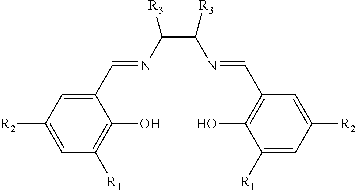

wherein R₁, R₂, R₃, M, and PL are described above.

Each R₁ may independently be selected from one or more of a 1,1 ethylphenyl propane, 2,2 methylphenyl ethane, 1,2-trimethyl propyl, 1,1-dimethylpropyl, 1-methylcyclohexyl, 1,1-diphenylethyl, or any combination thereof. Each R₂ may be independently selected from one or more of a methyl group, a C₂₋₁₆ alkyl group, an aryl group, an alkyl-aryl group, or any combination thereof. Each of the 3,5-substituted phenyls may be independently substituted at the 3 position by one or more of 1,1 ethylphenyl propane, 2,2 methylphenyl ethane, 1,2-trimethyl propyl, 1,1-dimethylpropyl, 1-methylcyclohexyl, 1,1-diphenylethyl, or any combination thereof. The metal carbonyl anion may be (Co(CO)₄). The ethyldiimine bridge may form a portion of one or more cyclohexane, a cyclohexene, or aromatic rings. The hydrocarbyl-diimine bridge may include an ethyldiimine that may be optionally substituted. The cyclohexane, cyclohexene, aromatic rings may be optionally substituted by one or more methyl groups, a C₂₋₁₀ alkyl group, hydroxyl, methoxy, trifluoromethyl, halogen, or any combination thereof. The ethyldiimine bridge may be substituted at a 1 position, a 2 position, or both by one or more of a hydrogen group, a methyl group, a C₂₋₁₀ alkyl group, or any combination thereof. The metal may include one or more of Al or Cr. The polar ligand may include one or more of tetrahydrofuran, dioxane, diethyl ether, or any combination thereof. The composition may have catalytic activity with one or more epoxides to form one or more lactones. The ligand may have an aromatic ring at one or more of the 3 positions, the 5 positions, or any combination thereof. The ligand may have aromaticity at one or more of the 3 positions, the 5 positions, or any combination thereof.

The method may further include preparing the 3, 5-substituted salicylaldehydes connected by an hydrocarbyl-diimine bridge or the ligand by contacting a carbonyl di-substituted at the carbon with a hydrogen, a —C₁-C₁₂ alkyl group, or an aryl group with a Grignard reagent under such conditions so that a —C₁-C₁₂ alkyl group or an aryl group is added to the carbonyl and the oxygen atom converts to a tertiary alcohol to form a trisubstituted tertiary alcohol. The method may further include contacting the trisubstituted tertiary alcohol with methane sulfonic anhydride under such conditions so that an alkene is formed which is 1,1-disubstituted with two or more of a hydrogen, a —C₁-C₁₂ alkyl group, or an aryl group. The method may further include contacting a phenol which is optionally substituted at the 4 position with a hydrogen, methyl, a —C₂-C₁₂ alkyl group, an aryl group, a t-butyl alkyl group, a halogen, an amine, —CF₃, hydrocarbyl oxy group, or —NO₂ with the alkene which is 1,1-disubstituted with two or more of a hydrogen, a —C₁-C₁₂ alkyl group, or an aryl group in the presence of an acid catalyst under such conditions so that the alkene is added at the 2 position of the aromatic ring to form a phenol that is substituted at the 2 position with the alkene and which is optionally substituted at the 4 position. The method may further include contacting the 2-alkene phenol which is optionally substituted at the 4 position with a formaldehyde or a formaldehyde precursor in the presence of a Lewis acid catalyst and a base under such conditions so that a salicylaldehyde is formed which is substituted at the 3 position by the alkene and is optionally substituted at the 5 position by a hydrogen, methyl, a —C₂-C₁₂ alkyl group, an aryl group, a t-butyl alkyl group, a halogen, an amine, —CF₃, hydrocarbyl oxy group, or —NO₂. The method may further include contacting the 3,5-substituted salicylaldehyde with a diamine under such conditions so that the 3, 5-substituted salicylaldehydes connected by an hydrocarbyl-diimine bridge or the compound according to the formula above is formed.

The present disclosure provides carbonylation catalysts that possess high catalytic activity with ethylene oxide. The present disclosure provides carbonylation catalysts that have improved steric and/or electron qualities which improve reaction yields, reduce time of reaction, reduce side products, increase catalyst longevity, increase catalyst stability and are more easily recoverable after a carbonylation reaction. The present disclosure provides methods for readily and easily assembling the catalyst from basic compounds. The present disclosure provides methods for using the catalyst to form lactones from ethylene oxide and carbon monoxide with high yields.

### BRIEF DESCRIPTION

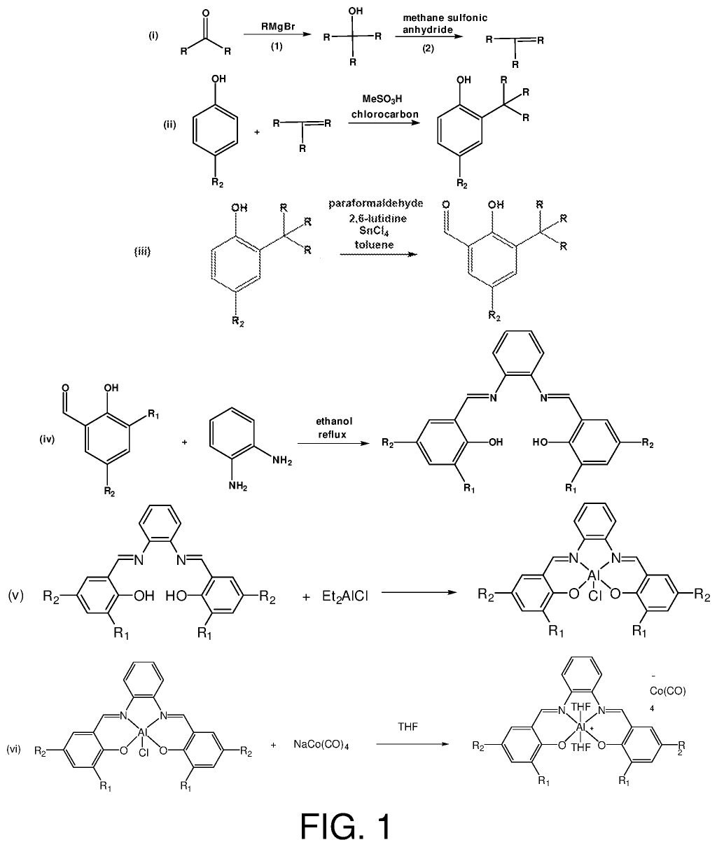

FIG. 1 is a synthetic scheme to form a carbonylation catalyst.

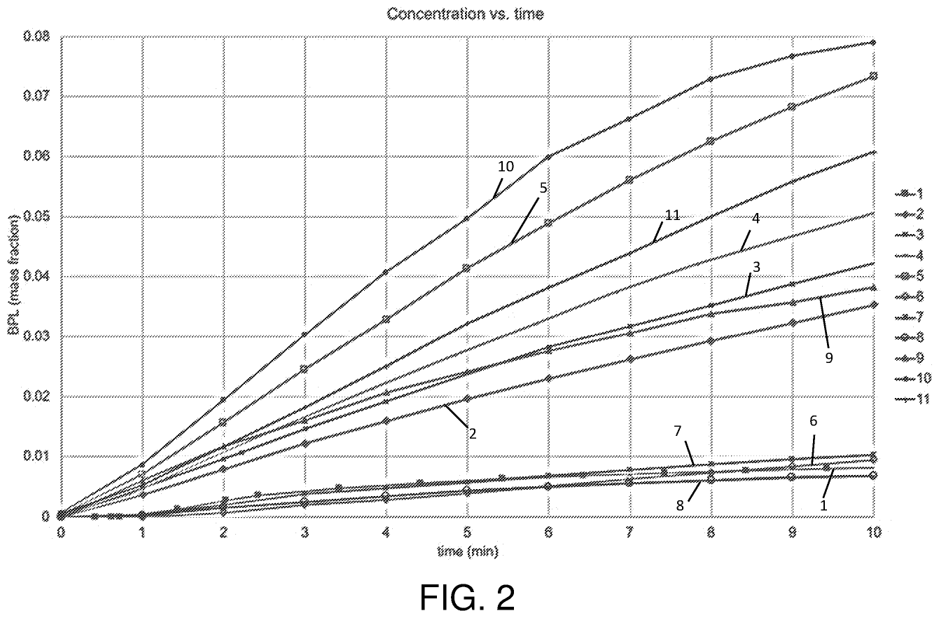

FIG. 2 illustrates beta propiolactone concentration versus time.

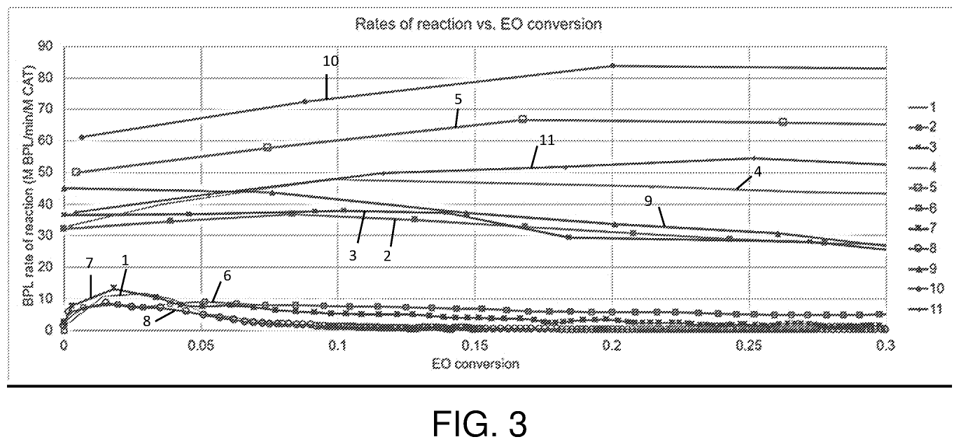

FIG. 3 illustrates the rate of formation of beta propiolactone in unites of M beta propiolactone/min/M catalyst up to 30 percent ethylene oxide conversion.

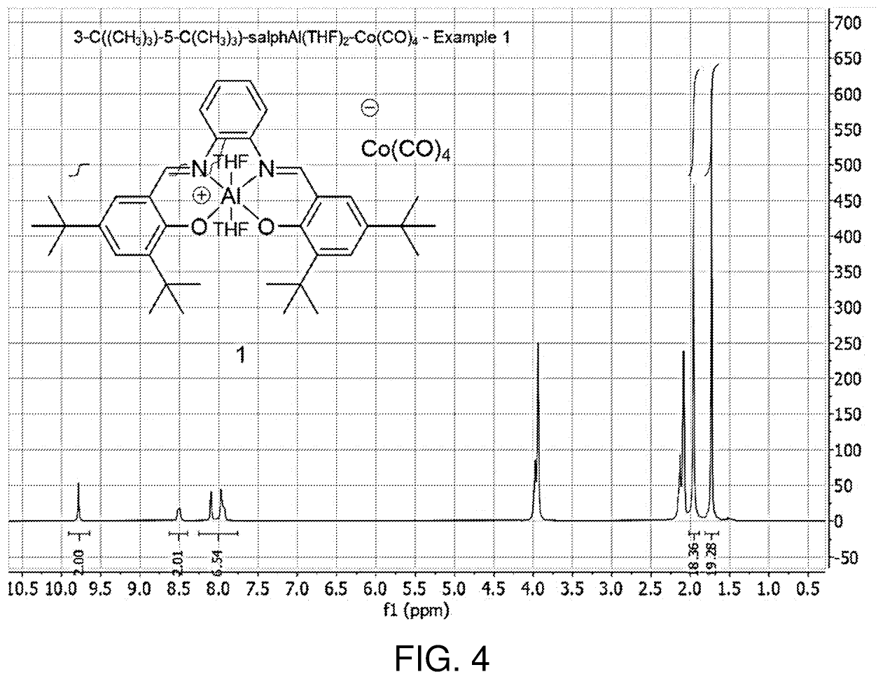

FIG. 4 is a 1H NMR spectrum of the isolated carbonylation catalyst, Example 1, analyzed in d8-THF solvent.

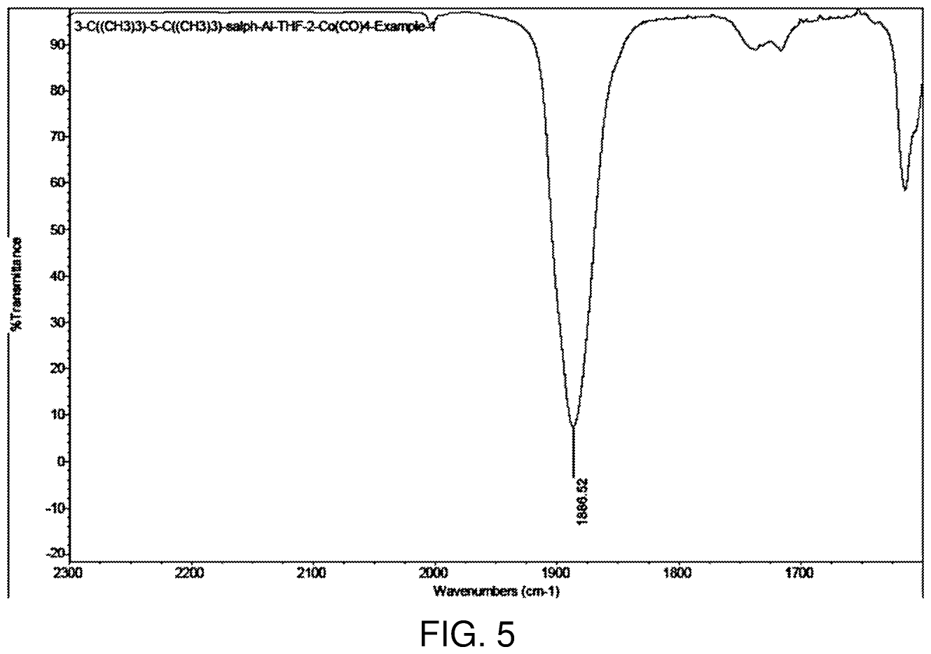

FIG. 5 is a FTIR spectrum of the isolated carbonylation catalyst, Example 1, depicting the carbonyl stretching peak associated with Co(CO)₄⁻ located at 1886 cm⁻¹ analyzed in THF solvent.

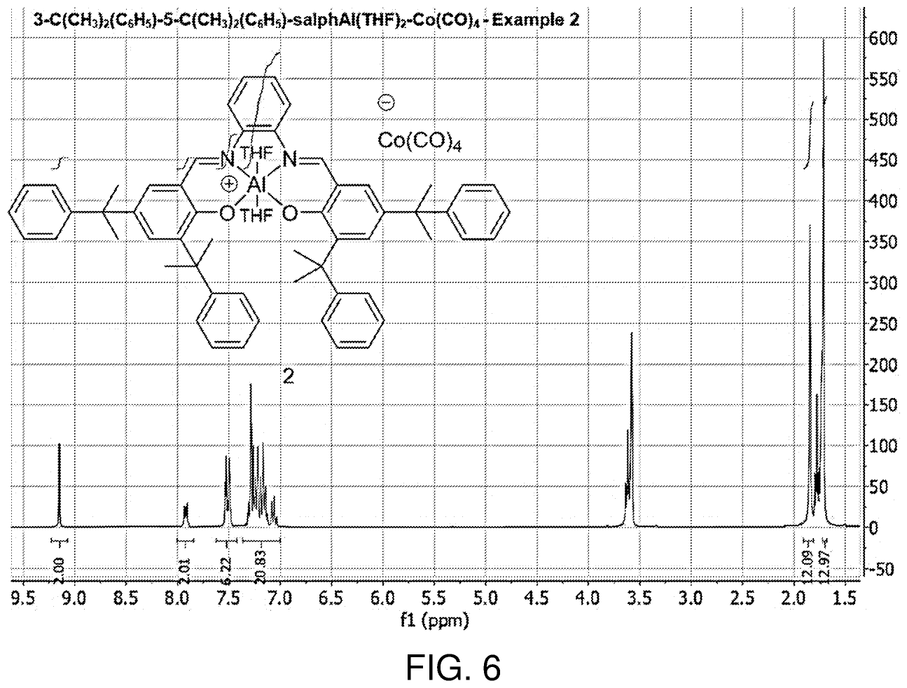

FIG. 6 is a 1H NMR spectrum of the isolated carbonylation catalyst, Example 2, analyzed in d8-THF solvent.

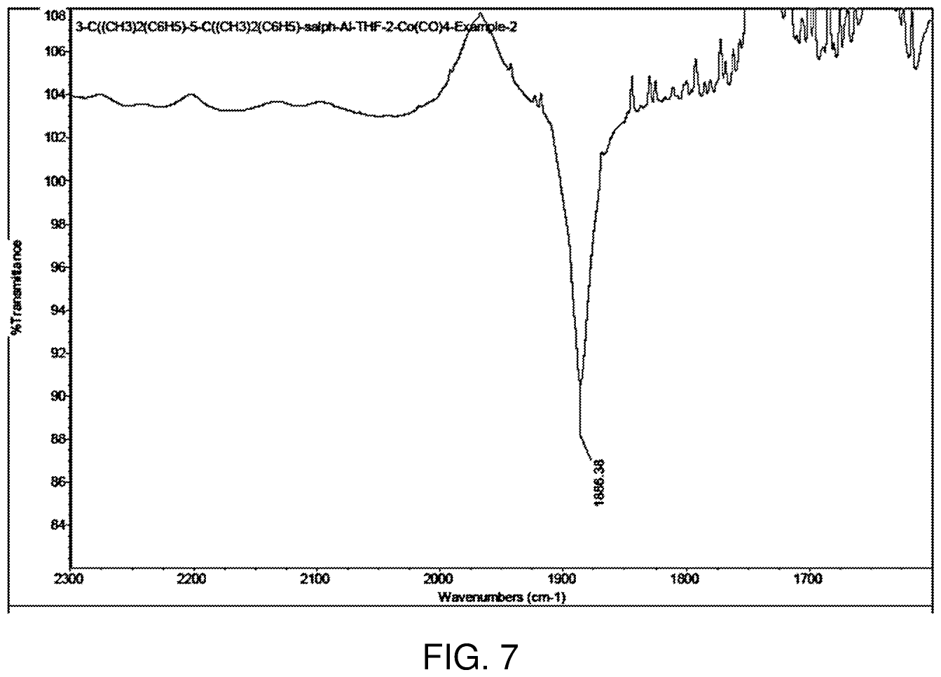

FIG. 7 is a FTIR spectrum of the isolated carbonylation catalyst, Example 2, depicting the carbonyl stretching peak associated with Co(CO)₄⁻ located at 1886 cm⁻¹ analyzed in THF solvent.

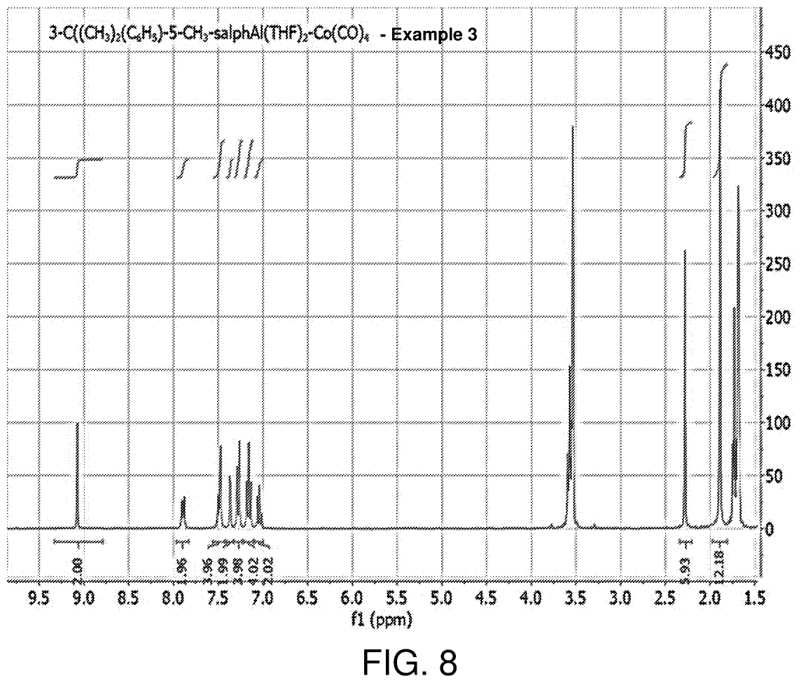

FIG. 8 is a 1H NMR spectrum of the isolated carbonylation catalyst, Example 3, analyzed in d8-THF solvent.

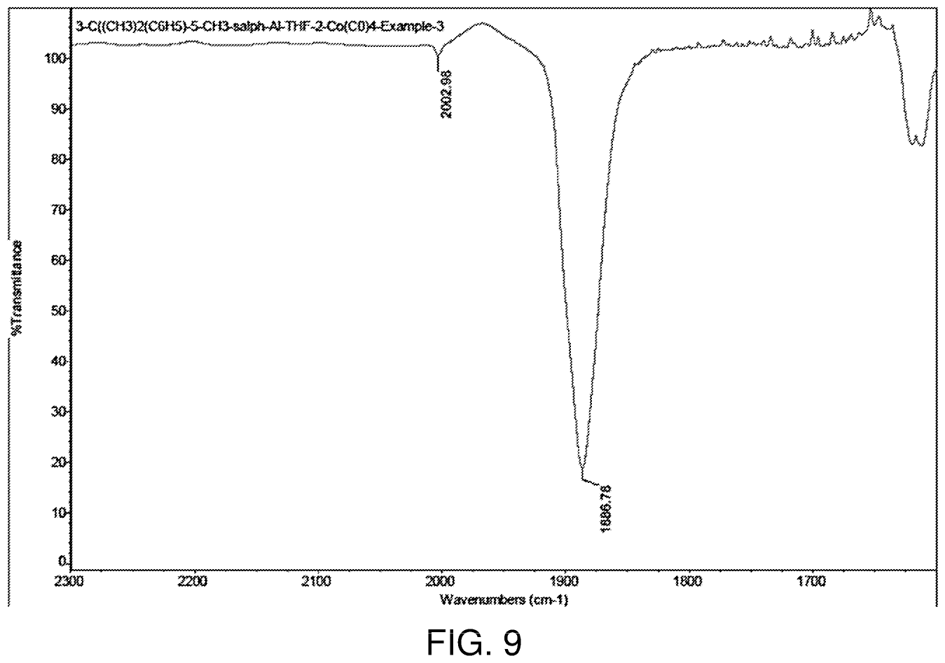

FIG. 9 is a FTIR spectrum of the isolated carbonylation catalyst, Example 3, depicting the carbonyl stretching peak associated with Co(CO)₄⁻ located at 1886 cm⁻¹ analyzed in THF solvent.

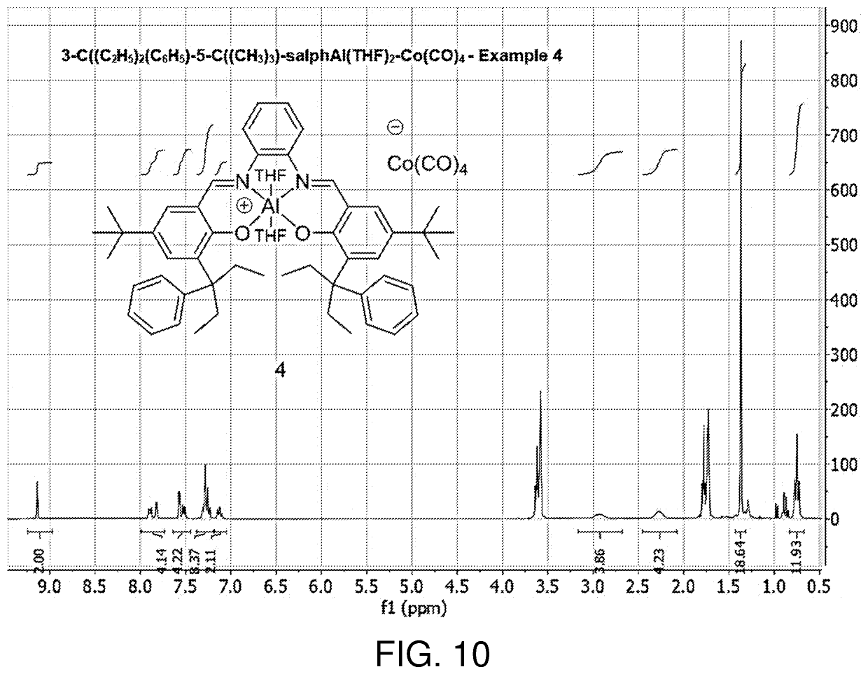

FIG. 10 is a 1H NMR spectrum of the isolated carbonylation catalyst, Example 4, analyzed in d8-THF solvent.

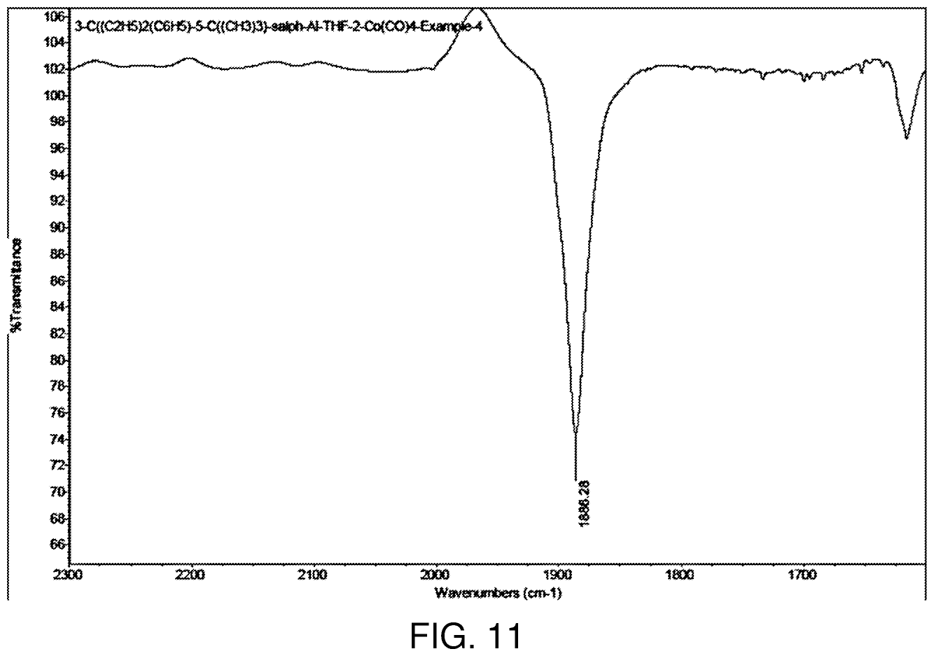

FIG. 11 is a FTIR spectrum of the isolated carbonylation catalyst, Example 4, depicting the carbonyl stretching peak associated with Co(CO)₄⁻ located at 1886 cm⁻¹ analyzed in THF solvent.

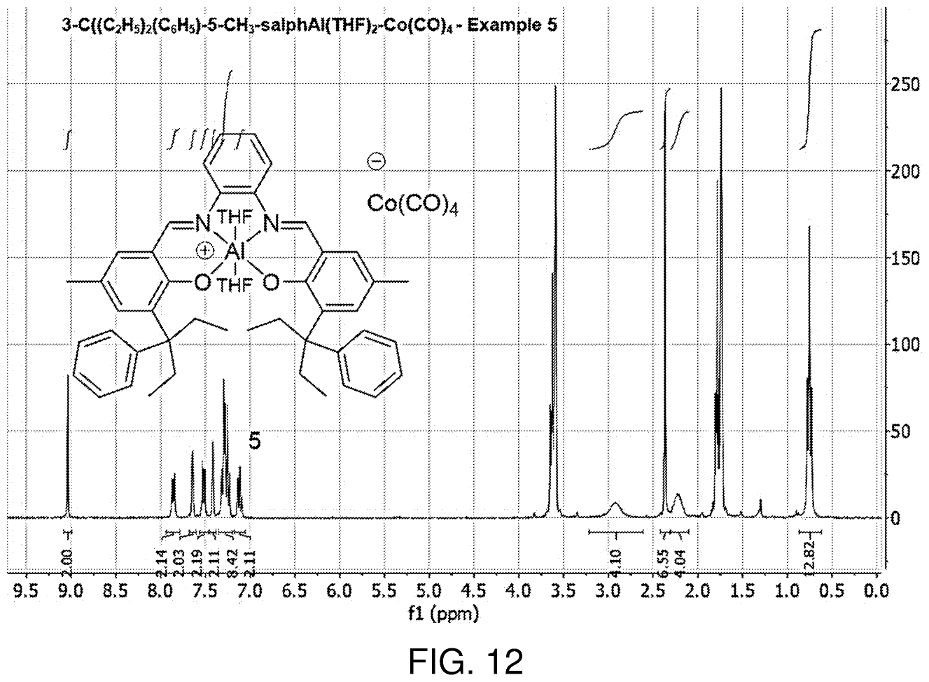

FIG. 12 is a 1H NMR spectrum of the isolated carbonylation catalyst, Example 5, analyzed in d8-THF solvent.

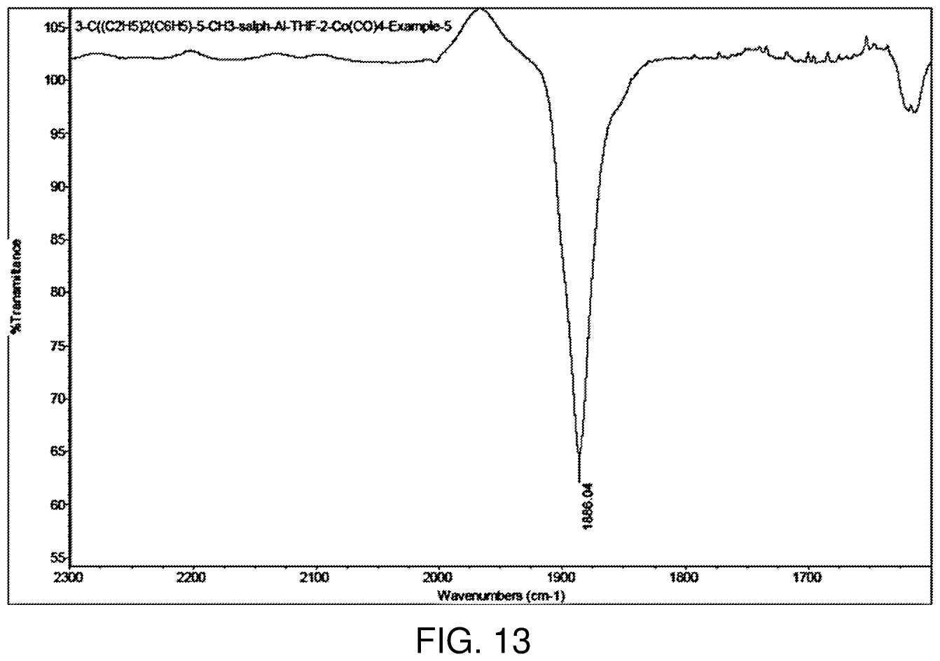

FIG. 13 is a FTIR spectrum of the isolated carbonylation catalyst, Example 5, depicting the carbonyl stretching peak associated with Co(CO)₄⁻ located at 1886 cm⁻¹ analyzed in THF solvent.

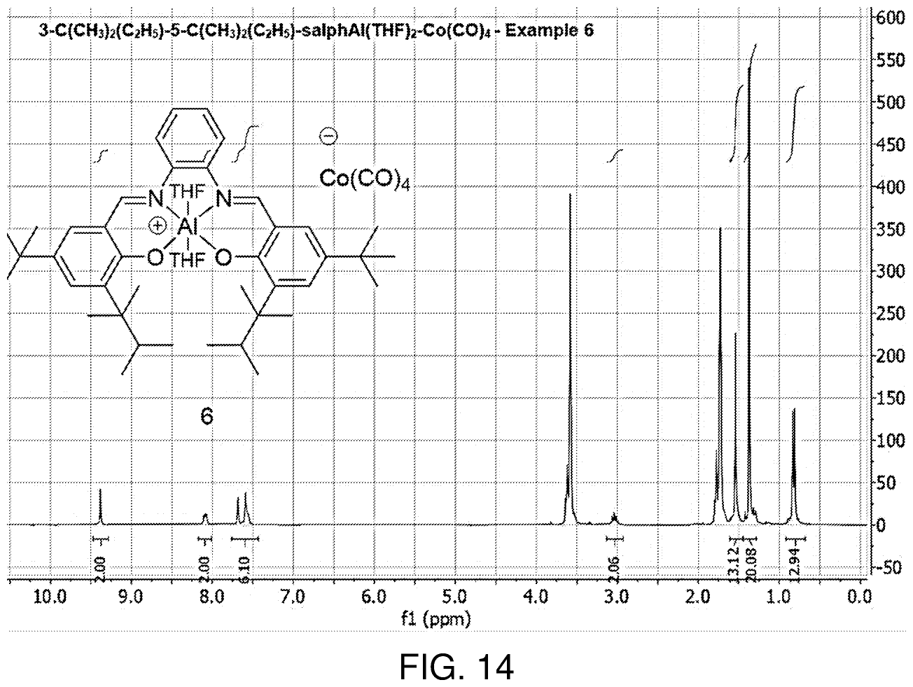

FIG. 14 is a 1H NMR spectrum of the isolated carbonylation catalyst, Example 6, analyzed in d8-THF solvent.

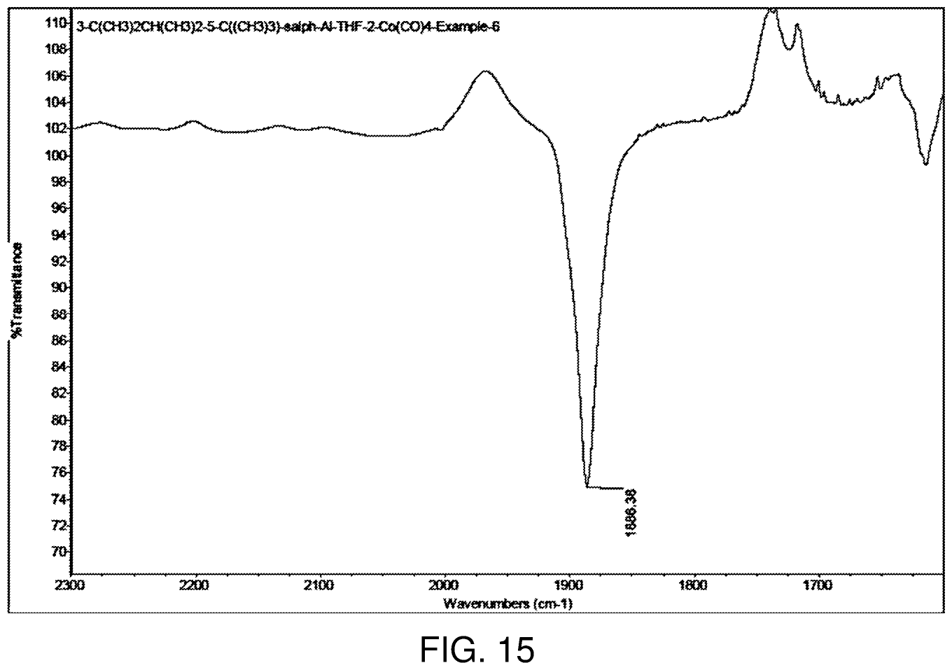

FIG. 15 is a FTIR spectrum of the isolated carbonylation catalyst, Example 6, depicting the carbonyl stretching peak associated with Co(CO)₄⁻ located at 1886 cm⁻¹ analyzed in THF solvent.

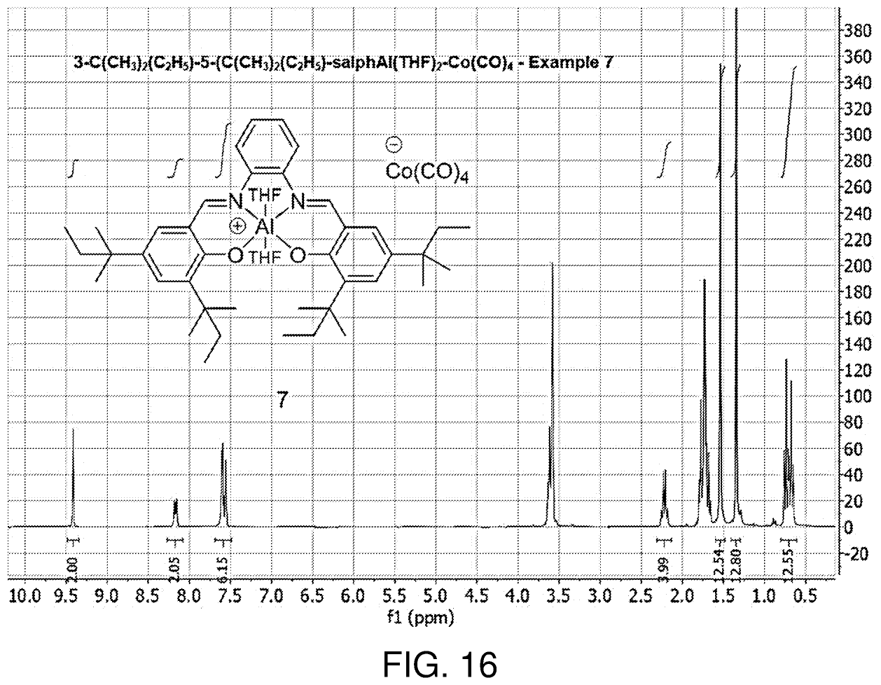

FIG. 16 is a 1H NMR spectrum of the isolated carbonylation catalyst, Example 7, analyzed in d8-THF solvent.

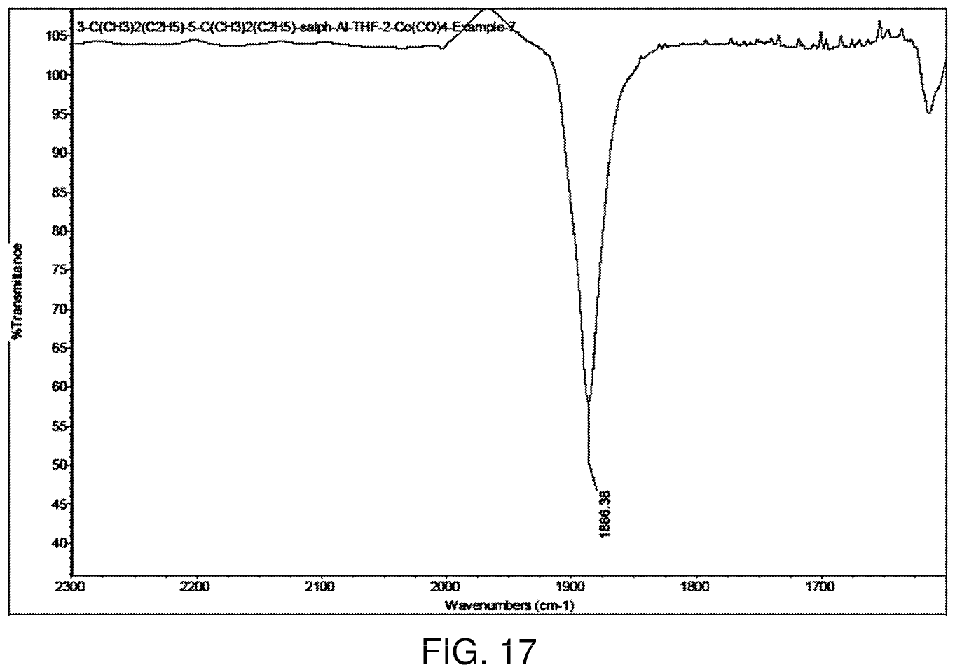

FIG. 17 is a FTIR spectrum of the isolated carbonylation catalyst, Example 7, depicting the carbonyl stretching peak associated with Co(CO)₄⁻ located at 1886 cm⁻¹ analyzed in THF solvent.

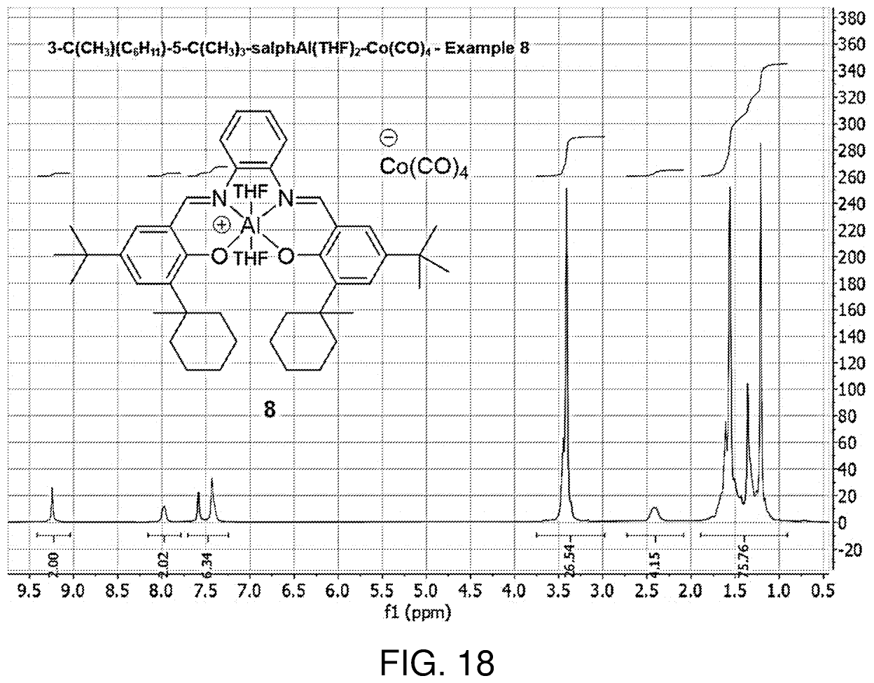

FIG. 18 is a 1H NMR spectrum of the isolated carbonylation catalyst, Example 8, analyzed in d8-THF solvent.

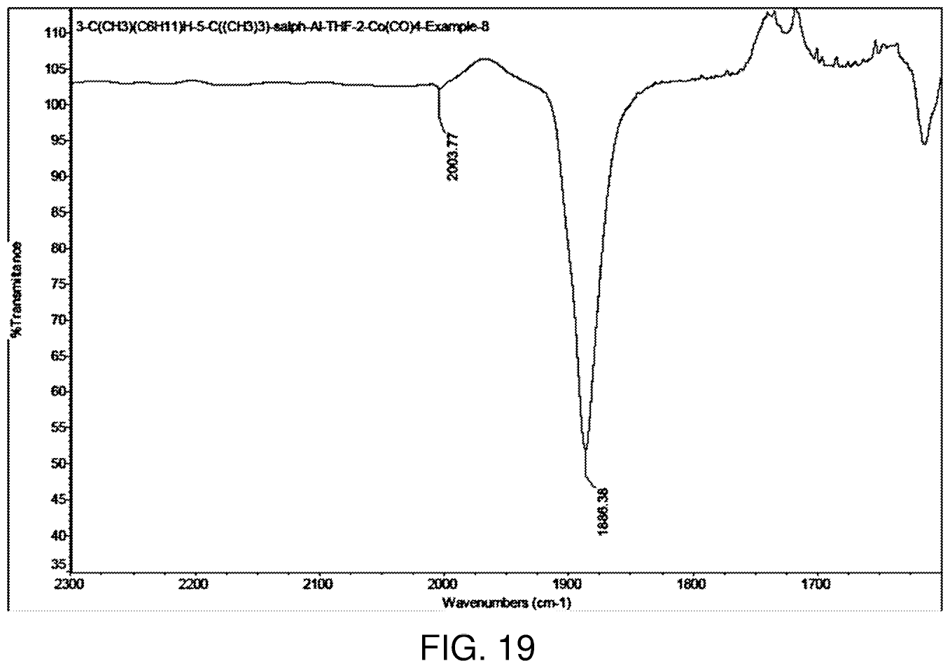

FIG. 19 is a FTIR spectrum of the isolated carbonylation catalyst, Example 8, depicting the carbonyl stretching peak associated with Co(CO)₄⁻ located at 1886 cm⁻¹ analyzed in THE solvent.

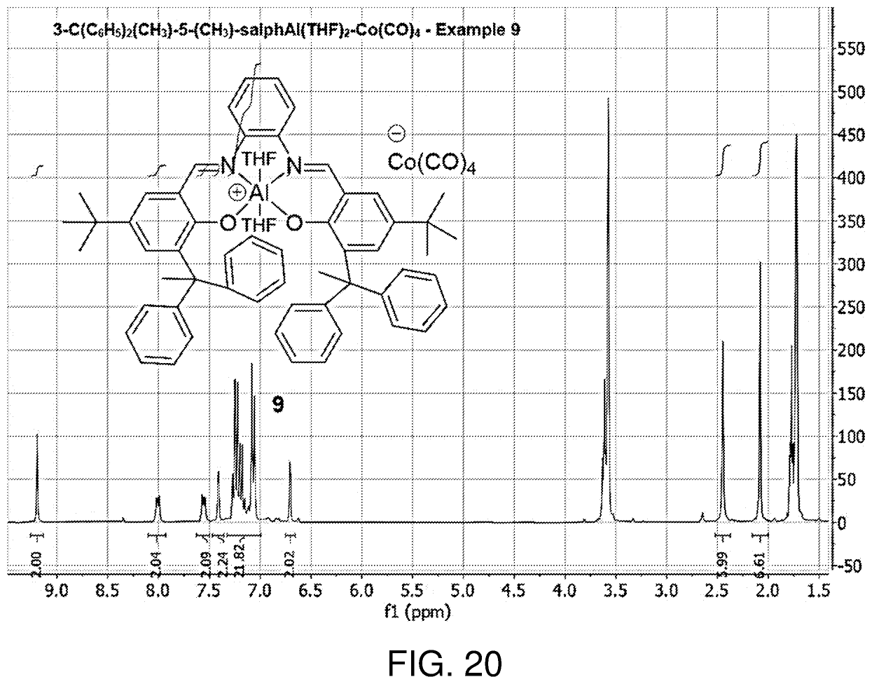

FIG. 20 is a 1H NMR spectrum of the isolated carbonylation catalyst, Example 9, analyzed in d8-THF solvent.

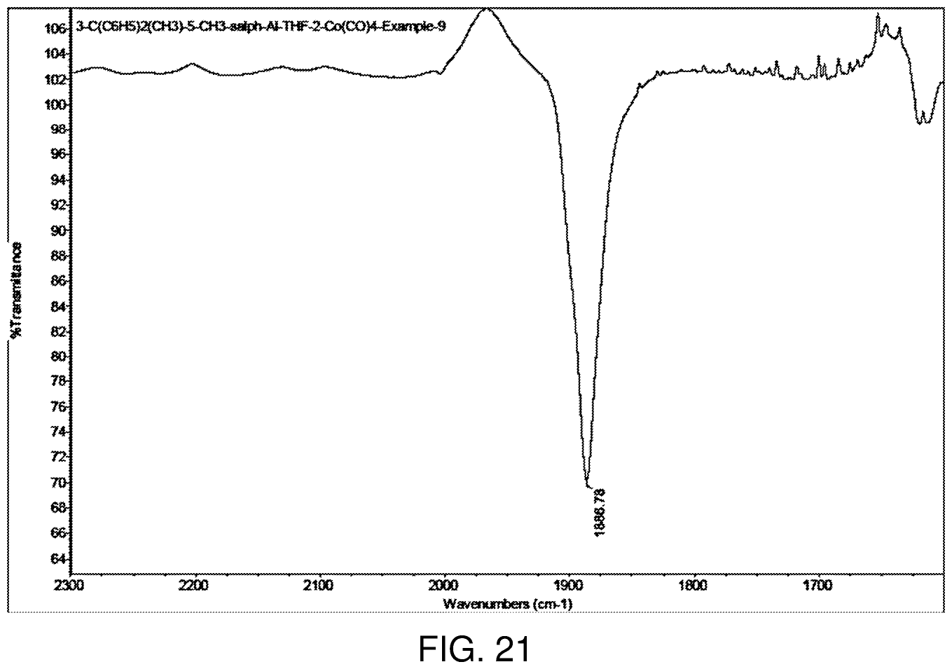

FIG. 21 is a FTIR spectrum of the isolated carbonylation catalyst, Example 9, depicting the carbonyl stretching peak associated with Co(CO)₄⁻ located at 1886 cm⁻¹ analyzed in THF solvent.

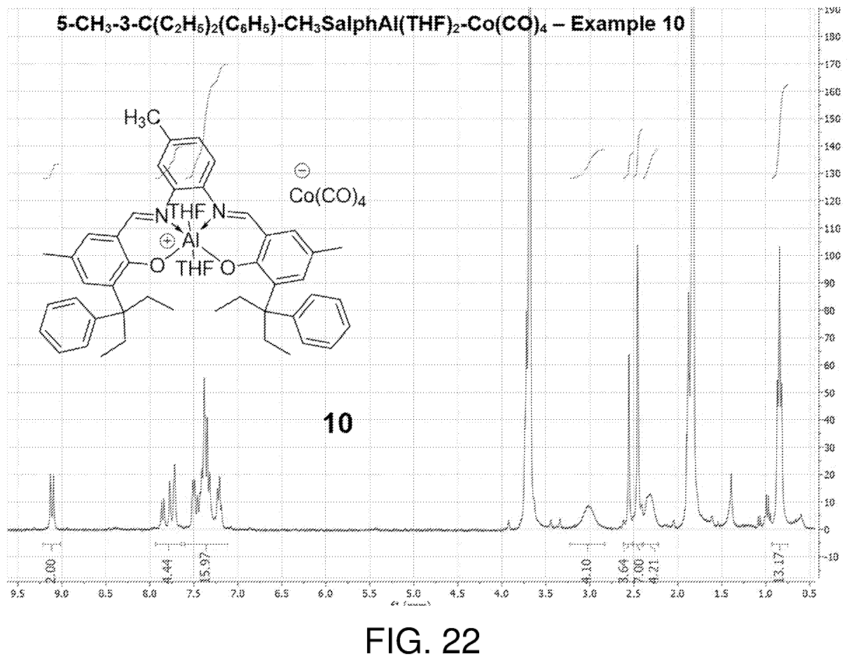

FIG. 22 is a 1H NMR spectrum of the isolated carbonylation catalyst, Example 10, analyzed in d8-THF solvent.

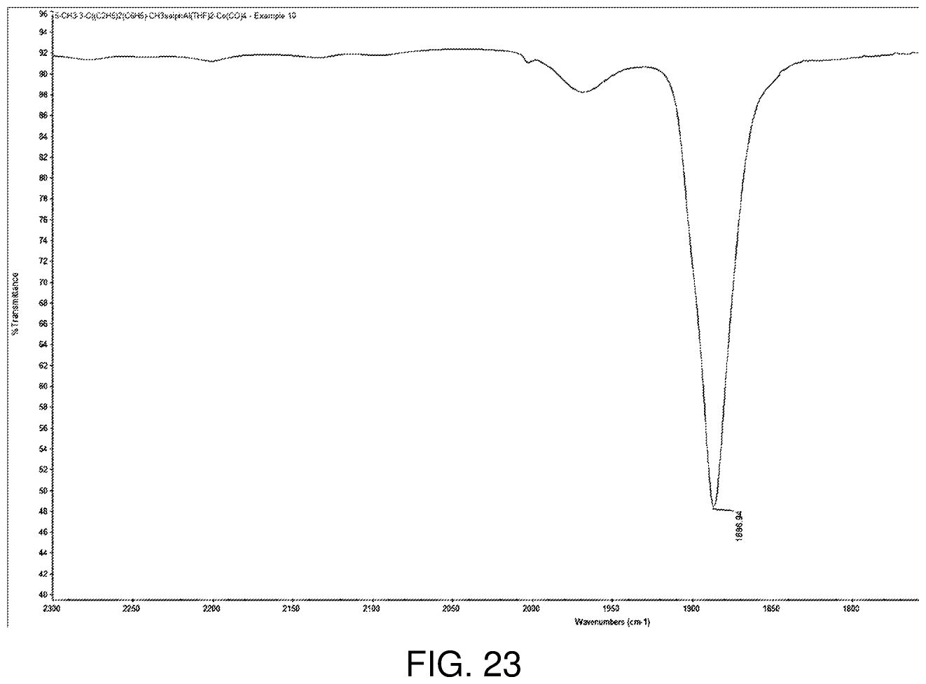

FIG. 23 is a FTIR spectrum of the isolated carbonylation catalyst, Example 10, depicting the carbonyl stretching peak associated with Co(CO)₄⁻ located at 1886 cm⁻¹ analyzed in THF solvent.

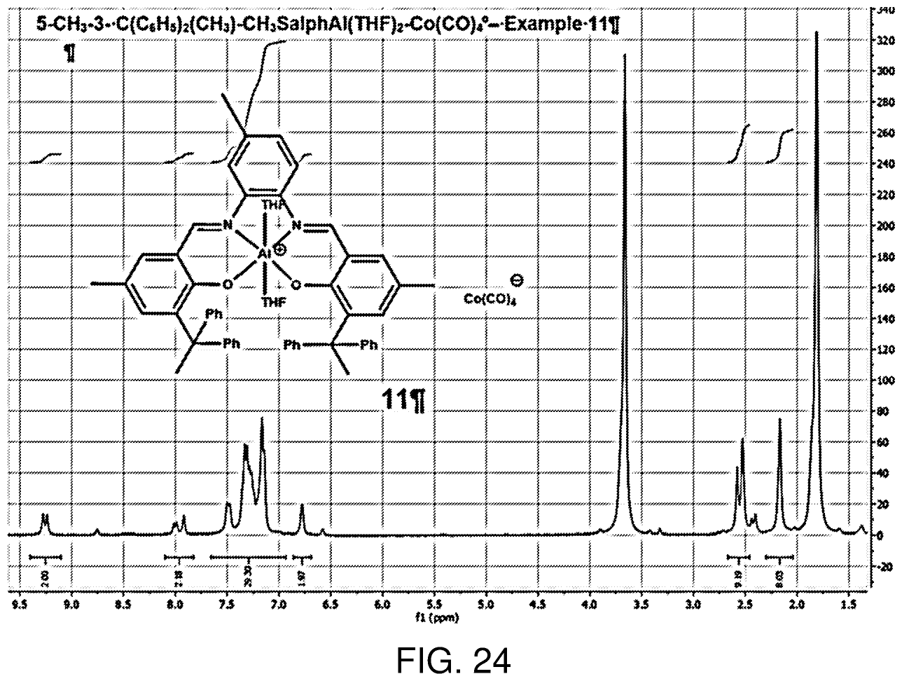

FIG. 24 is a 1H NMR spectrum of the isolated carbonylation catalyst, Example 11, analyzed in d8-THF solvent.

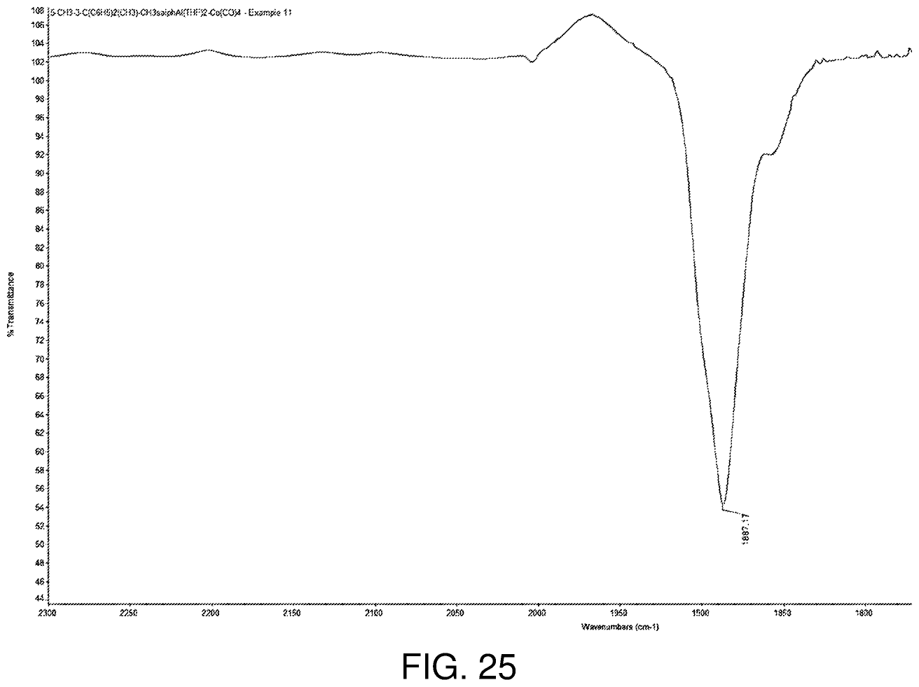

FIG. 25 is a FTIR spectrum of the isolated carbonylation catalyst, Example 11, depicting the carbonyl stretching peak associated with Co(CO)₄⁻ located at 1887 cm⁻¹ analyzed in THF solvent.

### DETAILED DESCRIPTION

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

One or more as used herein means that at least one, or more than one, of the recited components may be used as disclosed. Residue with respect to an ingredient or reactant used to prepare the polymers or structures disclosed herein means that portion of the ingredient that remains in the polymers or structures after inclusion as a result of the methods disclosed herein. Substantially all as used herein means that greater than 90 percent of the referenced parameter, composition, structure or compound meet the defined criteria, greater than 95 percent, greater than 99 percent of the referenced parameter, composition or compound meet the defined criteria, or greater than 99.5 percent of the referenced parameter, composition or compound meet the defined criteria. Portion as used herein means less than the full amount or quantity of the component in the composition, stream, or both. Precipitate as used herein means a solid compound in a slurry or blend of liquid and solid compounds. Phase as used herein means a solid precipitate or a liquid or gaseous distinct and homogeneous state of a system with no visible boundary separating the phase into parts. Parts per weight means parts of a component relative to the total weight of the composition per 100 parts of the composition. A catalyst component as used herein means a metal centered compound, a metal carbonyl, a Lewis acid, a Lewis acid derivative, a metal carbonyl derivative, or any combination thereof. A catalyst as used herein includes at least cationic compound and an anionic compound. An organic compound as used herein includes any compound that is free of a metal atom. An inorganic compound as used herein includes compounds that include at least one metal atom. Composition or mixture as used herein includes all components in a stream, reactant stream, product stream, slurry, precipitate, liquid, solid, gas, or any combination thereof that are containable within a single vessel. The phenols described herein may function to form the basis of one or more salicylaldehydes. Unsubstituted phenols may be formed by any known method, such as reaction of oxygen, benzene, and methyl-ethylene to form the phenol and acetone and further substituting the phenol using known methods. The phenols may be substituted at the 2 and/or 4 positions by one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof.

According to certain aspects, suitable hydrocarbyl groups can include at least straight or branched chain alkyl groups, straight or branched chain alkyl alkenyl groups, straight or branched chain alkynyl groups, cycloalkyl groups, alkyl substituted cycloalkyl groups, aryl groups, aralkyl groups, and alkaryl groups. Additionally, suitable hydrocarbyl groups can also contain one or more heteroatoms in the backbone of the hydrocarbyl group.

In certain aspects, a suitable hydrocarbyl group can also, or alternatively, be substituted with a substituent group. Non-limiting examples of substituent groups can include one or more alkyl, halo, alkoxy, alkylthio, hydroxyl, nitro, cyano, azido, carboxy, acyloxy, and sulfonyl groups. In certain aspects, substituent groups can be selected from one or more alkyl, halo, alkoxy, alkylthio, and hydroxyl groups. In certain aspects, substituent groups can be selected from one or more halo, alkyl, and alkoxy groups.

In certain aspects, suitable hydrocarbyl groups can be C₁₋₂₀ hydrocarbyl groups. For example, the hydrocarbyl group can be an alkyl ether having one or more alkyl ether groups or alkylene oxy groups. Suitable alkyl ether groups can include, without limitation, ethoxy, propoxy, and butoxy groups. In certain aspects, suitable hydrocarbyl groups can contain about 1 to about 100 alkylene oxy groups; in certain aspects, about 1 to about 40 alkylene oxy groups; and in certain aspects, about 1 to about 10 alkylene oxy groups. In certain aspects, suitable hydrocarbyl groups can contain one or more heteroatoms in the backbone.

Suitable examples of more specific hydrocarbyl groups can include, in certain aspects, C₁₋₁₅ straight or branched chain alkyl groups, C₁₋₁₅ straight or branched chain alkenyl groups, C₅₋₁₈ cycloalkyl groups, C₆₋₂₄ alkyl substituted cycloalkyl groups, C₄₋₁₈ aryl groups, C₄₋₂₀ aralkyl groups, and C₄₋₂₀ alkaryl groups. In certain aspects, the hydrocarbyl group can more preferably be C₁₋₈ straight or branched chain alkyl groups, C₁₋₂ cycloalkyl groups, C₆₋₁₂ alkyl substituted cycloalkyl groups, C₄₋₁₈ aryl groups, C₄₋₂₀ aralkyl groups, or C₄₋₂₀ alk-aryl groups.

As used herein, alkaryl can include an alkyl group bonded to an aryl group. Aralkyl can include an aryl group bonded to an alkyl group. Aralkyl can also include alkylene bridged aryl groups such as diphenyl methyl or propyl groups. As used herein, aryl can include groups containing more than one aromatic ring. Cycloalkyl can include groups containing one or more rings including bridge rings. Alkyl substituted cycloalkyl can include a cycloalkyl group having one or more alkyl groups bonded to the cycloalkyl ring.

In certain aspects, suitable alkyl groups can include methyl, ethyl, propyl, isopropyl, butyl, tertiary butyl, pentyl, hexyl, and ethyl hexyl. Similarly, examples of suitable cycloalkyl groups can include cyclohexyl and fenchyl and examples of suitable alkyl substituted groups can include menthyl and isobornyl.

According to certain aspects, suitable hydrocarbyl groups can include methyl, ethyl, propyl, isopropyl, butyl, tertiary butyl, ethyl pentyl, hexyl, ethyl hexyl, fenchyl, menthyl, and isobornyl groups.

Disclosed herein are compounds and methods useful as carbonylation catalysts that have improved steric properties so that reaction with epoxide is improved to form lactones, reaction with lactones are avoided, and isomerization of the epoxide to aldehydes or ketones is avoided. The processes described herein to make the carbonylation catalysts provide a novel way to synthesize 3,5-disubstituted salicylaldehydes, which are precursors to the carbonylation catalysts, with desirable substituted groups that improve the efficiency of the carbonylation reaction. Additionally, the synthetic methods herein allow for integration of varying groups at the hydrocarbyl-diimine bridge between 3,5-salicylaldehydes, which can provide additional steric and/or electronic properties that improve the conversion rate of epoxide to lactone. The compositions and associated ligands of the cations may include aromaticity that improves the overall efficiency of the conversion of epoxide to beta propiolactone due to electron and/or steric considerations.

Disclosed herein are compositions that may include a metal carbonyl anion and a cation ionically bonded to the metal carbonyl anion. The cation may include a ligand having two residues of 3,5-substituted salicylaldehydes connected by an hydrocarbyl-diimine bridge that includes a nitrogen atom contacted with a carbon of an aldehyde residue at each of the two residues of the 3,5-substituted salicylaldehydes. Each of the residues of the 3,5-substituted salicylaldehydes may be independently substituted at one or both of a 3 position and a 5 position by a hydrocarbyl group containing at least 5 carbons. The cation may include a metal coordinated with the ligand at each hydroxyl residue and the two residues of the 3,5-substituted salicylaldehydes at a 2 position and at each of the nitrogen atoms of the hydrocarbyl-diimine bridge. The cation may include two polar ligands coordinated with the metal. The 3,5-substituted salicylaldehydes may be substituted at any other position that does not impair the function of the carbonylation catalyst.

The ligands of the cation of the carbonylation catalyst may have the following structure 1:

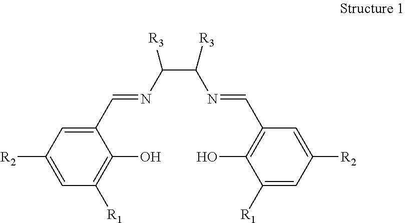

wherein each R₁ is independently selected from one or more of an alkyl group, an aryl group, or an alkyl-aryl group;
wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
wherein at least one of R₁ or R₂ is selected from an alkyl group, an aryl group, or an alkyl-aryl group that contains at least 5 carbons; and
wherein each R₃ is independently selected from hydrogen, methyl, a C₂₋₁₀ alkyl group, a combination thereof, or in combination form a six membered ring that is optionally a substituted aromatic ring.

After a metalation step, the cation of the carbonylation catalyst may have the following structure 2:

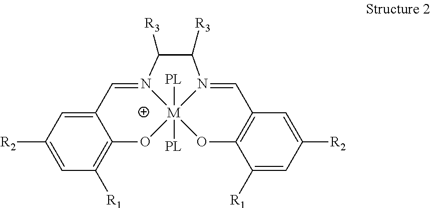

wherein each PL is a polar ligand, such as tetrahydrofuran, dioxane, diethyl ether, any other metal described herein, or any combination thereof;
wherein M is a metal; such as aluminum, chromium, or any other metal described herein;
   wherein each R₁ is independently selected from one or more of an alkyl group, an aryl group, or an alkyl-aryl group;
   wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
   wherein at least one of R₁ or R₂ is selected from an alkyl group, an aryl group, or an alkyl-aryl group that contains at least 5 carbons; and
   wherein each R₃ is independently selected from hydrogen, methyl, a C₂₋₁₀ alkyl group, a combination thereof, or in combination form a six membered ring that is optionally a substituted aromatic ring.

After combination with an anion, such as a metal carbonyl, the carbonylation catalyst may have the following structures 3-12:

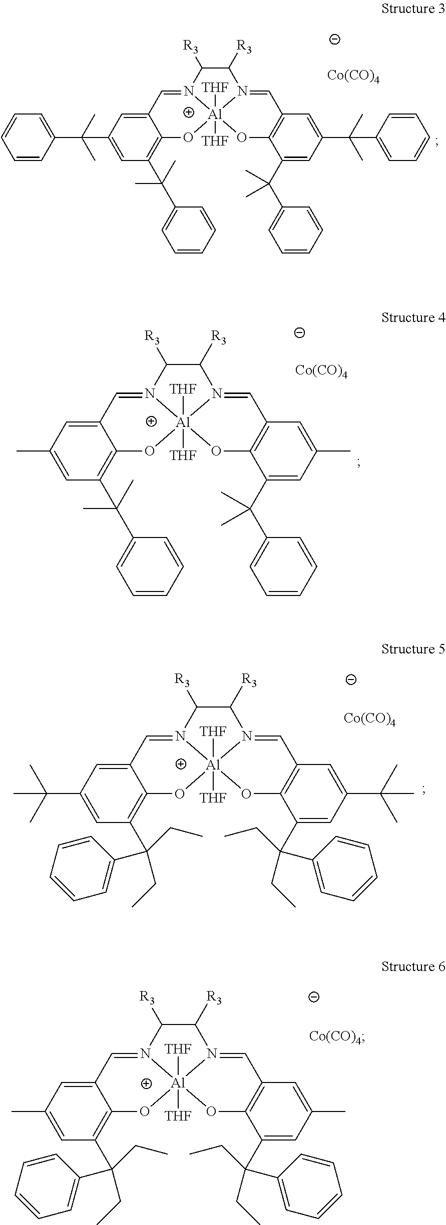

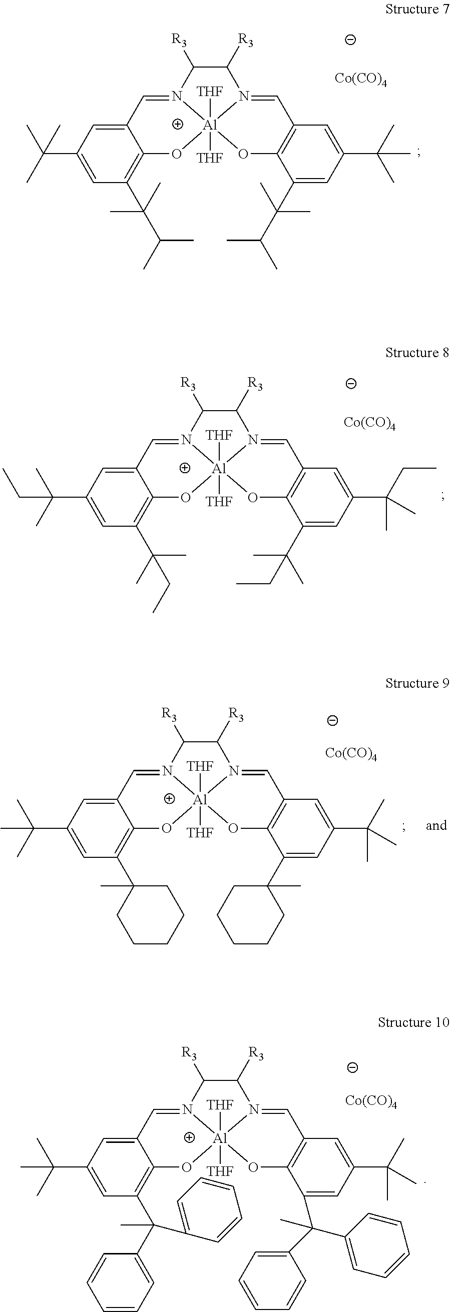

wherein each R₃ is independently selected from hydrogen, methyl, a C₂₋₁₀ alkyl group, a combination thereof, or in combination form a six membered ring that is optionally a substituted aromatic ring.

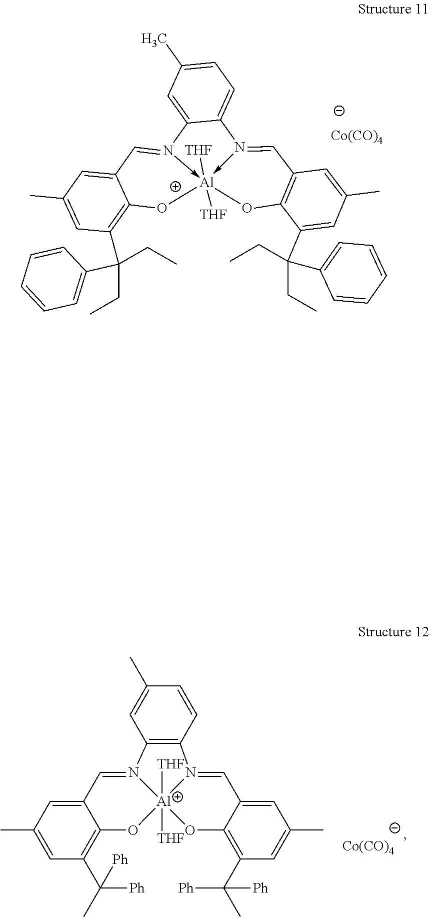

wherein Ph comprises a phenyl group.

Structures 11 and 12 may have a different hydrocarbyl-diimine bridge. For example, the hydrocarbyl-diimine bridge may be an ethyldiimine bridge that may be optionally substituted. The hydrocarbyl-diimine bridge may form a portion of an aromatic ring that may have a different substitution or may not be substituted, a cyclohexane ring that may be optionally substituted, or a cyclohexene ring that may be optionally substituted.

Structures 1-12 may be substituted at any other position that does not impair the functionality of the carbonylation catalyst.

The carbonylation catalysts described herein may be used to react carbon monoxide and an epoxide to form a lactone, as described herein. The carbonylation catalysts described herein may also be used to react an aziridine and carbon monoxide to form a lactam.

The carbonylation reaction may include contacting one or more epoxides, lactones, or both with carbon monoxide in the presence of catalyst. This step may occur in a reactor that has one or more inlets, two or more inlets, three or more inlets, or a plurality of inlets. The one or more epoxides, the lactones, the carbon monoxide, and the catalyst may be added in a single inlet, multiple inlets, or each may be added in a separate inlet as separate or combined feed streams. The carbonylation reaction may produce one or more product streams or compositions.

The epoxide used in the carbonylation reaction may be any cyclic alkoxide containing at least two carbon atoms and one oxygen atom. For example, the epoxide may have a structure shown by formula (I):

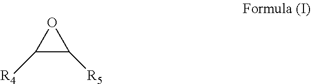

where R₄ and R₅ are each independently selected from the group consisting of: hydrogen; C₁-C₁₅ alkyl groups; halogenated alkyl groups; phenyl groups; optionally substituted aliphatic or aromatic alkyl groups; optionally substituted phenyl; optionally substituted heteroaliphatic alkyl groups; optionally substituted 3 to 6 membered carbocycle; and optionally substituted 3 to 6 membered heterocycle groups, where R₄ and R₅ can optionally be taken together with intervening atoms to form a 3 to 10 membered, substituted or unsubstituted ring optionally containing one or more hetero atoms; or any combination thereof.

The lactone formed from the carbonylation reaction may be any cyclic carboxylic ester having at least one carbon atom and two oxygen atoms. For example, the lactone may be an acetolactone, a propiolactone, a butyrolactone, a valerolactone, caprolactone, or a combination thereof. Anywhere in this application where a propiolactone or lactone is used or described, another lactone may be applicable or usable in the process, step, or method. Where a propiolactone is a used or produced in the carbonylation reaction, the propiolactone may have a structure corresponding to formula II:

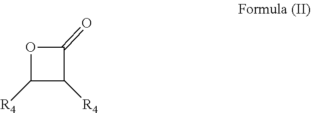

where R₄ and R₅ are each independently selected from the group consisting of: hydrogen; C₁-C₁₅ alkyl groups; halogenated alkyl groups; phenyl groups; optionally substituted aliphatic or aromatic alkyl groups; optionally substituted phenyl; optionally substituted heteroaliphatic alkyl groups; optionally substituted 3 to 6 membered carbocycle; and optionally substituted 3 to 6 membered heterocycle groups, where R₄ and R₅ can optionally be taken together with intervening atoms to form a 3 to 10 membered, substituted or unsubstituted ring optionally containing one or more hetero atoms; or any combination thereof.

The product stream or composition may include one or more organic compounds including a propiolactone, a polypropiolactone, succinic anhydride, polyethylene glycol, poly-3-hydroxypropionate, 3-hydroxy propionic acid, 3-hydroxy propionaldehyde, a polyester, a polyethylene, a polyether, unreacted epoxides, any derivative thereof, any other monomer or polymer derived from the reaction of an epoxide and carbon monoxide, or any combination thereof. The product stream or composition may include one or more inorganic compounds that include catalyst components such as metal carbonyls, metal carbonyl derivatives, metal centered compounds, Lewis acids, Lewis acid derivatives, or any combination thereof. A metal carbonyl derivative is a compound that includes one or more metals and one or more carbonyl groups that can be processed to form an anionic metal carbonyl component for use in a carbonylation catalyst. A Lewis acid derivative is a compound that includes one or more metal centered Lewis acids bonded to one or more undesirable compounds at the metal center that can be processed into a cationic Lewis acid for use in a carbonylation catalyst. The product stream or composition may include a catalyst that has not been spent or used up in the process of forming propiolactones. The product stream or composition may include one or more unreacted epoxides or carbon monoxide.

To make the alkyl, aryl, or alky-aryl substituted groups of the salicylaldehydes of the carbonylation catalysts described herein, a carbonyl that is di-substituted may be contacted with a Grignard reagent in a Grignard reaction. The Grignard reagent may function to facilitate adding an alkyl or aryl group to another compound. The Grignard reagent may be any compound sufficient to facilitate movement of an alkyl or aryl group to form an alcohol, such as a tertiary alcohol. The Grignard reagent may have the structure of R—Mg—X, where X is a halogen and R is an organic group. The Grignard reagent may be selected from one or more of alkyl magnesium bromide, aryl magnesium bromide, or a combination of both. The carbonyl may be di-substituted at the carbon with a hydrogen, a —C₁-C₁₂ alkyl group, or an aryl group. The carbonyl that is disubstituted at the carbon may function to form the basis for an R₁ or R₂ group on a ligand, a 3,5-disubstituted salicylaldehyde, a 4-substitute phenol, and/or a 2,4-disubstituted phenol. The carbonyl may be any carbonyl sufficient to bond with a phenol group. The carbonyl may include a ketone or an aldehyde. The carbonyl may have a structure according to R₁—CO—R₂, where each R₁ or R₂ may be independently selected from hydrogen atom, a methyl group, a C₂₋₁₆ alkyl group, an aryl group, an alkyl-aryl group, a halogen, an amine, a trifluoromethyl group, a nitro group, a hydrocarbyl oxy group.

The Grignard reaction may add a —C₁-C₁₂ alkyl group or an aryl group to the carbonyl and the oxygen atom converts to a tertiary alcohol to form a trisubstituted tertiary alcohol. The carbonyl may be first dissolved in a polar solvent, such as dichloromethane, and cooled. Relative to polarity, the polar solvent is sufficient to add an alkyl or aryl group to carbonyl. The combination of carbonyl and polar solvent may be cooled to any temperature sufficient to add an alkyl or aryl group and convert the oxygen atom to a tertiary alcohol and, thus, form a trisubstituted tertiary alcohol. For example, the temperature may be about 15 degrees Celsius or less, about 10 degrees Celsius or less, or about 5 degrees Celsius or less. The temperature may be cooled to a temperature of about-15 degrees Celsius or more, about-10 degrees Celsius or more, or about-5 degrees Celsius or more. Slowly, over a period of time, the Grignard reagent may be added to the polar solvent and the carbonyl. The Grignard reagent may be added to polar solvent and the carbonyl over any period of time and in any molar ratio sufficient to form the trisubstituted tertiary alcohol. The Grignard reagent may be added in excess relative to the carbonyl to assist with adding the alkyl or aryl group to the carbonyl. For example, the Grignard reagent and the carbonyl may be contacted in a molar ratio of about 1:1 or greater, 1.3:1 or greater, or about 1.5:1 or greater. The Grignard reagent and the carbonyl may be contacted in a molar ratio of about 2:1 or less, about 1.8:1 or less, or about 1.6:1 or less. The period of time may be about 30 minutes or less, about 20 minutes or less, or about 15 minutes or less. The period of time may be about 5 minutes or more, about 8 minutes or more or about 10 minutes or more. The reaction mixture of carbonyl, polar solvent, and Grignard reagent may then be warmed to room temperature and stirred for a period of time. The Grignard reagent may be stirred for any amount of time and warmed to any temperature sufficient to form the trisubstituted tertiary alcohol. The period of time may be about 12 hours or more, about 18 hours or more, or about 24 hours or more. The period of time may be about 48 hours or less, about 36 hours or less, or about 30 hours or less. After stirring, the reaction may be cooled any temperature sufficient to add an acidic solution. The reaction may be cooled to about-15 degrees Celsius or more, about-10 degrees Celsius or more, or about-5 degrees Celsius or more. After stirring, the reaction may be cooled to about 15 degree Celsius or less, about 10 degrees Celsius or less, or about 5 degrees Celsius or less. After cooling, an acidic solution may be added to form an organic and an aqueous phase. The acidic solution may contain saturated ammonium chloride. The acidic solution may have any acidity sufficient to form an alkene from a tertiary alcohol. For example, the acidic solution may be fully saturated. In other examples, the acidic solution may be free of a protic acid. The organic phase may be separated from the aqueous phase by extraction or any other known technique, and the organic phase may be dried, such as by sodium sulfate or magnesium sulfate. The solvent of the organic phase may be removed under reduced pressure or any other known technique and the trisubstituted tertiary alcohol may be collected. The trisubstituted tertiary alcohol may be further purified by column chromatography on silica gel.

After isolation and purification, the trisubstituted tertiary alcohol may be converted to a 1,1-disubstituted alkene for attachment with a substituted phenol. The trisubstituted tertiary alcohol may be dissolved in a polar solvent to form a mixture in an inert atmosphere, such as under a nitrogen stream (i.e., free of ambient air and/or moisture). This may be done in a Schlenk line or a dry box, which are free of air and moisture. The mixture may be cooled to a temperature of about-15 degrees Celsius or more, about-10 degrees or more, or about-5 degrees or more. The trisubstituted tertiary alcohol dissolved in the polar solvent may be cooled to about 15 degrees Celsius or less, about 10 degrees Celsius or less, or about 5 degrees Celsius or less. After cooling, a base is contacted with the trisubstituted tertiary alcohol in the polar solvent. The base may function to perform an elimination reaction by abstracting a proton from a OH group of a tertiary alcohol after it has been activated by a methane sulfonic anhydride. Exemplary bases may include one or more of 2,6-lutidine, triethylamine, or both.

The mixture may then be stirred for any period of time sufficient to abstract the proton from the OH group of the tertiary alcohol. The mixture may be stirred for about 30 seconds or more, about 45 seconds or more, or about 1 minute or more. The mixture may be stirred for about 1.75 minutes or less, about 1.50 minutes or less, or about 1.25 minutes or less. After stirring, methane sulfonic anhydride that is dissolved in a polar solvent is slowly contacted with the mixture of the trisubstituted tertiary alcohol, the polar solvent, and the base over a period of time. The methane sulfonic anhydride may function to form an alkene from a tertiary alcohol. In lieu of methane sulfonic anhydride, any other component sufficient to participate in an elimination reaction. For example, phosphorous oxychloride or toluene sulfonic anhydride may be used in lieu of methane sulfonic anhydride. The methane sulfonic anhydride may be contacted with the trisubstituted tertiary alcohol and the base for any period of time sufficient to form an alkene. For example, the components may be contacted for about 10 minutes or less, about 8 minutes or less, or about 6 minutes or less. The methane sulfonic anhydride may be contacted with the mixture over about 2 minutes or more, about 3 minutes or more, or about 5 minutes or more. The base, the methane sulfonic anhydride, and the trisubstituted tertiary alcohol may be contacted in any molar ratio sufficient to form an alkene from the trisubstituted tertiary alcohol. For example, the base, methane sulfonic anhydride, and the trisubstituted tertiary alcohol may be contacted in a molar ratio of about 4:2:1 or more, about 3:2:1 or more, or about 3:3:2 or more. After addition of the methane sulfonic anhydride to the mixture, the overall mixture may be stirred for any period of time sufficient to form the alkene. For example, the overall mixture may be stirred for about 0 degrees Celsius for about 30 minutes or more, about 40 minutes or more, or about 50 minutes or more. The overall mixture may be stirred at about 0 degrees Celsius for about 90 minutes or less, about 80 minutes or less, or about 70 minutes or less. Subsequently, brine and water may be added to the overall mixture to separate the mixture into aqueous and organic layers. The brine may be a fully saturated solution. The organic layer may be separated from the aqueous layer and concentrated under reduced pressure. The organic layer comprises the 1,1-disubstituted alkene and may be purified by dissolving the organic layer in a nonpolar solvent that may be aprotic and passing the organic layer over a frit with a layer of silica gel.

After forming the 1,1-disubstituted alkene and the 4-substituted phenol, the 1,1-disubstituted alkene and the 4-substituted phenol may be combined to form a 2,4-disubstituted phenol. The 1,1-disubstituted alkene and the 4-substituted phenol may be contacted in a polar solvent, and an acid catalyst is added dropwise to form a mixture. The acid catalyst may function to combine a 4-substituted phenol and an alkene and can be any acid sufficient to assist with combination of the alkene and the 4-substituted phenol at the 2 position. Exemplary acid catalysts include methane sulfonic acid, H₂SO₄, phosphoric acid, any other mineral acid, or any combination thereof. The 1,1-disubstituted alkene, the 4-substituted phenol, and the methane acid catalyst may be contacted in any molar ratio sufficient to add the 1,1-disubstited alkene to the 4-substituted phenol. For example, the molar ratio may be about 2:1.5:1 or greater. The mixture may stir for any period of time sufficient to add the 1,1-disubstituted alkene to the 4 substituted phenol at the 2 position. For example, the mixture may stir for about 8 hours or more, about 12 hours or more, or about 16 hours or more. The mixture may stir for about 28 hours or less, about 20 hours or less, or about 16 hours or less. After stirring, water and a polar aprotic solvent may be added to the mixture to form an aqueous and an organic layer. The organic layer may be extracted, and the 2, 4 disubstituted phenol may be isolated by any known technique. For example, the organic layer may be washed with a base reagent, such as saturated sodium bicarbonate, and dried with a drying agent, such as sodium sulfate or magnesium sulfate. After washing and drying, the 2,4 disubstituted phenol is purified by column chromatography over silica gel.

After forming and/or isolating the 2,4-disubstituted phenol, the 2,4-disubstituted phenol may be contacted with formaldehyde or formaldehyde precursor to make a 2,4,6-trisubstiuted phenol having an aldehyde at the 6 position. Under an inert atmosphere (i.e., under a nitrogen atmosphere, such as in a Schlenk line or dry box), the 2,4-disubstituted phenol and a base may be contacted in a polar aprotic solvent to form a reaction mixture. Exemplary solvents include those disclosed herein after. The reaction mixture may be reduced to a temperature at which room temperature is reached. For example, the temperature may be reduced to a temperature of about-15 degrees Celsius or more, about-10 degrees Celsius or more, or about −5 degrees Celsius or more. The reaction mixture may be reduced to temperature of about 15 degrees Celsius or less, about 10 degrees Celsius or less, or about 5 degrees Celsius or less. After reducing the temperature, a Lewis acid catalyst may be slowly added to the reaction mixture through a septum. The Lewis acid catalyst may function to assist with forming the aldehyde at the 6 position of the 2,4-disubsituted phenol. Exemplary Lewis acid catalysts may be selected from one or more of SnCl₄, AlCl₃, FeCl₃, or any combination thereof. The reaction mixture may be allowed to stir at room temperature (i.e., about 25 degrees Celsius) for any period of time sufficient to assist with forming the aldehyde at the 6 position of the 2,4-disubsituted phenol. For example, the reaction mixture may stir at room temperature for about 5 minutes or more, about 10 minutes or more, or about 15 minutes or more. The reaction mixture may be allowed to stir for about 30 minutes or less, about 25 minutes or less, or about 20 minutes or less.

After stirring, formaldehyde or a formaldehyde precursor may be added to the mixture, and the overall mixture may be placed under a slight vacuum (i.e., >1 atm). The formaldehyde or formaldehyde precursor may function to add an aldehyde group to a 2,4-disubstituted phenol. The formaldehyde or the formaldehyde precursor may be any compound sufficient to add an aldehyde group to a 2,4-disubstituted phenol at the 6 position. The formaldehyde or the formaldehyde precursor may be paraformaldehyde. While under slight vacuum, the overall mixture may be heated and stirred for a period of time. The mixture may be heated to any temperature and stirred for any period of time sufficient to form a precursor to the 2,4,6 disubstituted phenol. For example, the overall mixture may be heated to about 75 degrees Celsius or more, about 80 degrees Celsius or more, or about 85 degrees Celsius or more. The mixture may be heated to about 105 degrees Celsius or less, about 100 degrees Celsius or less, or about 95 degrees Celsius or less. The overall mixture while being heated may be stirred for about 8 hours or more, about 10 hours or more, or about 12 hours or more. The overall mixture may be heated for about 24 hours or less, about 20 hours or less, or about 16 hours or less.

Once the period of time for stirring is expired, the overall mixture may be cooled to room temperature (i.e., about 25 degrees Celsius), and an acid may be added to the overall mixture and stirred for any period of time sufficient to finalize adding an aldehyde to the 2,4 disubstituted phenol at the 6 position. The acid may be include any acid sufficient to cleave the Lewis Acid catalyst at the oxygen residue of the phenol at the 1 position and quench any unreacted Lewis acid catalyst. For example, the acid may be HCl, phosphoric acid, sulfuric acid, any other mineral acid, or any combination thereof. For example, regarding temperature, the reaction may be stirred for about 45 minutes or more, about 50 minutes or more, or about 55 minutes or more. The overall mixture may be stirred for 75 minutes or less, about 70 minutes or less, or about 65 minutes or less. The 2,4,6-trisubstiuted phenol having an aldehyde at the 6 position may be formed at this stage in the overall mixture and may be isolated or purified using any known technique. For example, a polar aprotic solvent may be added to the overall mixture to form aqueous and organic layers. The organic layer may then be extracted and washed with sodium bicarbonate and brine. The brine may be a fully saturated solution. After washing, the organic layer may be dried with a drying agent such as sodium sulfate or magnesium sulfate. The organic solvent of the organic layer may be removed under reduced pressure to yield the desired 2,4,6-trisubstiuted phenol having an aldehyde at the 6 position.

After forming and isolating the 2,4,6-trisubstiuted phenol having an aldehyde at the 6 position, the 2,4,6-trisubstiuted phenol having an aldehyde at the 6 position may be subjected to a condensation step to form a ligand containing two residues of 3,5-substituted salicylaldehydes connected by an hydrocarbyl-diimine bridge that includes a nitrogen atom contacted with a carbon of an aldehyde residue at each of the two residues of the 3,5-substituted salicylaldehydes. For example, the ligand may have a structure according to structure 1. In this step, the 2,4,6-trisubstiuted phenol having an aldehyde at the 6 position may be contacted with a diamine in the presence of a Lewis acid or Bronsted acid and a alkali metal or ammonium salt in the a polar solvent to form the desired ligand containing two residues of 3,5-substituted salicylaldehydes connected by an hydrocarbyl-diimine bridge. In other examples, the reaction may be free of a Lewis acid or Bronsted acid so that the diamine and the 2,4,6-trisubstituted phenol may be contacted in a polar solvent. These steps may be completed in ambient air and may be completed at room temperature (e.g., about 25 degrees Celsius) or may be completed at reflux of the polar solvent. The condensation reaction may be conducted for a sufficient time period such that a ligand structure is formed containing two 3,5-disubstituted salicylaldehyde and a diamine. The diamine may function to bridge two 3,5-disubstituted salicylaldehydes to form a ligand. The diamine may be any compound including at least an ethyl bridged between two amine groups. The diamine may form a portion of a cyclohexane or cyclohexene ring. The diamine may include substituted groups. The diamine may be selected from one or more of ethylene diamine, ortho-phenylene diamine, ortho-cyclohexyl diamine, phenanthroline, bipyridine, substituted ethylene diamine, substituted phenylene diamine, substituted ortho-cyclohexyl diamine, substituted phenanthroline, or substituted bipyridine.

For example, the condensation reaction may persist for about 6 hours or more, about 10 hours or more, or about 14 hours or more. The condensation reaction may be conducted for about 28 hours or less, 24 hours or less, or about 20 hours or less. The 2,4,6-trisubstituted phenol and the diamine, may be added in a molar ratio to a ligand structure is formed containing two 3,5-disubstituted salicylaldehyde and a diamine, for example, of about 1:2 or more. The Lewis acid or Bronsted acid may be added in an amount sufficient to catalyze the condensation reaction. The Lewis acid or Bronsted base may include formic acid, acetic acid, any other carboxylic acid, or a combination thereof. The ligand two residues of 3,5-substituted salicylaldehydes connected by an hydrocarbyl-diimine bridge may be isolated from the product composition by any known technique or any technique described herein. For example, the ligand two residues of 3,5-substituted salicylaldehydes connected by an hydrocarbyl-diimine bridge may be collected as a precipitate by any known means of separating solids from liquids such as gravity filtration after the product composition has cooled.

After forming ligand containing the residues of two 3, 5-substituted salicylaldehydes connected by an hydrocarbyl-diimine bridge at the aldehyde residue of the two residues of the 3,5-substituted salicylaldehydes, the ligand may be subjected to a metalation step to form a metal centered compound containing a halogen or an alkyl group. The halogen or the alkyl group of the metal centered compound may be bonded to the metal center of the metal centered compound. In the metalation step, a metal alkyl compound may be contacted with the ligand in a nonpolar solvent at room temperature to form a metal centered compound containing a halogen or alkyl group. The metal alkyl compound and the ligand may be contacted together in any molar ratio sufficient to form the ligand. For example, the molar ratio may be about 1:1. The metalation step may be stirred for any amount of time sufficient to form the metal centered compound containing the halogen or the alkyl group. For example, the metalation step may be stirred for about 36 hours or less, about 30 hours or less, or about 24 hours or less. The metalation step may be stirred for about 12 hours or more, about 18 hours or more, or about 22 hours or more. The metalation step may be conducted under an inert gas, such as nitrogen, and in a dry box and/or Schlenk line. The metalation step may be conducted in open air or may be conducted in an inert atmosphere free of oxygen and water, such as a dry box or Schlenk line. The metalation step may be similar to the metalation steps described in U.S. Pat. No. 8,633,123, incorporated herein by reference in its entirety. After the metalation step is complete, the metal centered compound containing the halogen or the alkyl group may be isolated using any known technique, such as collecting the metal centered compound containing the halogen or the alkyl group by gravity filtration. The steps to form the metal centered compound containing the halogen or alkyl group may be performed under conditions that are moisture and oxygen free, for example, under an inert gas, like nitrogen, in a dry box or Schlenk line.

After forming and isolating one of the metal centered compound containing the halogen or the alkyl group above, the metal centered compound containing the halogen or the alkyl group may be subjected to a catalyst formation step to form the carbonylation catalyst. The catalyst formation step may include contacting the metal centered compound containing the halogen or the alkyl group with a polar ligand, a metal carbonyl additive, or both to from the carbonylation catalyst. The metal centered compound containing the halogen or alkyl group may be added in a molar ratio of about 1:1. The metal carbonyl additive may contain at least a metal carbonyl that is anionic and a cationic group that is configured to cleave and bond with the alkyl group or the halogen of the metal centered compound. The cationic group may be one or more of an alkali metal, any counterion sufficient to ionically bond and/or balance the metal carbonyl, or any combination thereof. In examples where the metal carbonyl additive cleaves or decouples the alkyl group, the alkyl group may couple with the cationic group, and the alkyl group and cationic group could be removed via any filtration or removal means described herein. In examples where the metal carbonyl additive cleaves the halogen from the metal centered compound and is contacted with the polar compound, the halogen bonds with the cationic group of the metal carbonyl additive and the metal centered compound containing the polar compound is formed. Any byproducts can be removed by any other removal or separation steps described herein. After the metal carbonyl additive cleaves or decouples the alkyl group, the metal centered compound may combine with the polar ligand to form a cationic species. The metal centered compound containing the polar ligand then contacts with the anionic metal carbonyl of the metal carbonyl additive and forms the regenerated carbonylation catalyst.

The steps to form the carbonylation catalyst may be performed under conditions that are moisture and oxygen free. For example, the catalyst formation steps may be performed within a dry glove box, on a Schlenk line, or in a reactor under an inert atmosphere (i.e., nitrogen). The catalyst formation steps may be performed under a nitrogen, argon, or any other inert gas. During the catalyst formation steps, the metal centered compound, the polar ligand, the metal carbonyl, or any combination thereof may be contacted and agitated by stirring for a period of time sufficient to form the carbonylation catalyst. The period of time for stirring the components may be about 5 minutes or more, about 30 minutes or more, about 60 minutes or more. The period of time for stirring the components may be about 24 hours or less, about 12 hours or less, or about 6 hours or less. The components in the catalyst formation steps may be completed under ambient temperature and/or pressure. Additional steps to make the regenerated catalyst can be found in U.S. Pat. No. 6,852,865B2 and U.S. Pat. No. 8,481,756B1, both of which are included herein by reference in their entirety.

The filtering, isolating, or removing steps taught herein function to remove from the composition any unwanted components that may interfere with the formation of a carbonylation catalyst or any precursor of the carbonylation catalyst. For example, one or more of solvents, polymers, unreacted acid compounds, inorganic compounds, organic compounds, or any combination thereof may be removed from the composition so that the carbonylation catalyst may be regenerated from the metal centered compound containing a halogen or an alkyl compound and have catalytic activity with one or more of succinic anhydride, propiolactone, or an epoxide. The filtering, isolating, or removing steps may include one or more of vacuum filtration, gravity filtration, centrifugation, decantation, precipitation, phase layer extraction, or any combination thereof. The filtering, isolating, or removing steps may utilize any method sufficient to separate one or more of solvents, polymers, unreacted acid compounds, inorganic compounds, organic compounds, or any combination thereof and the metal centered compound containing the halogen or a alkyl group, the ligands, or any combination thereof. The filtering, isolating, or removing steps may remove a single type of compound at a time, such as a precipitate, or may remove a collection of compounds at a time, such as all components dissolved in a solvent. The filtering or removing steps may include forming multiple phases including one or more of one or more organic phases, an aqueous phase, a solid phase (i.e., a precipitate), one or more gaseous or vapor phases, or any combination thereof. The one or more separation or removal steps/methods described herein may be performed at any temperature, pressure, agitation rate, time, or any combination thereof sufficient to separate or remove any undesirable component from the composition including the ligands, the metal centered compound containing the halogen or alkyl group, or any combination thereof.

The carbonylation catalyst as described herein functions to catalyze a reaction of an epoxide and carbon monoxide to produce one or more propiolactones and other products. The carbonylation catalyst includes at least a metal carbonyl that is anionic and a metal centered compound that is cationic.

The metal carbonyl of the carbonylation catalyst functions to provide the anionic component of the carbonylation catalyst. The carbonylation catalyst may include one or more, two more, or a mixture of metal carbonyls. The metal carbonyl may be capable of ring-opening an epoxide and facilitating the insertion of CO into the resulting metal carbon bond. In some examples, the metal carbonyl may include an anionic metal carbonyl moiety. In other examples, the metal carbonyl compound may include a neutral metal carbonyl compound. The metal carbonyl may include a metal carbonyl hydride or a hydrido metal carbonyl compound. The metal carbonyl may be a pre-catalyst which reacts in situ with one or more reaction components to provide an active species different from the compound initially provided. The metal carbonyl includes an anionic metal carbonyl species. in some examples, the metal carbonyl may have the general formula [Q_{d}M′ₑ(CO)_{w}]^{y-}, where Q is an optional ligand, M′ is a metal atom, d is an integer between 0 and 8 inclusive, e is an integer between 1 and 6 inclusive, w is a number such as to provide the stable anionic metal carbonyl complex, and y is the charge of the anionic metal carbonyl species. The metal carbonyl may include monoanionic carbonyl complexes of metals from groups 5, 7 or 9 of the periodic table or dianionic carbonyl complexes of metals from groups 4 or 8 of the periodic table. The metal carbonyl may contain cobalt, manganese, ruthenium, or rhodium. Exemplary metal carbonyls may include [Co(CO)₄]⁻, [Ti(CO)e]²⁻, [V(CO)₆]⁻, [Rh(CO)₄]⁻, [Fe(CO)_{4]}²⁻, [Ru(CO)_{4]}²⁻, [Os(CO)_{4]}⁻², [Cr₂(CO)₁₀]²⁻, [Fe₂(CO)₈]²⁻, [Tc(CO)₅]⁻, [Re(CO)₅]⁻, and [Mn(CO)₅]⁻. The metal carbonyl may be a mixture of two or more anionic metal carbonyl complexes in the carbonylation catalysts used in the methods.

The metal alkyl compound may function to coordinate a metal in one or more ligands to form a metal centered compound containing a halogen or an alkyl group. The metal alkyl compound may be any compound containing a metal and/or one or more alkyl groups and/or halogen group. The metal of the metal alkyl compound may be one or more of aluminum, chromium, or any combination thereof. The meal alkyl compound may include one or more of CrCl₂, (Et)₂AlCl or (Et)₃Al, or any combination thereof.

A metal carbonyl additive functions to deliver a metal carbonyl to a metal centered compound that is suitable to combine and form the carbonylation catalyst. The metal carbonyl additive may function to decouple a halogen or an alkyl group from a metal centered compound to form the carbonylation catalyst that includes the metal centered compound and metal carbonyl combination. The metal carbonyl additive includes at least a metal carbonyl as described herein and a cationic compound. The cationic compound may include lithium, sodium, potassium, rubidium, cesium, beryllium, magnesium, calcium, strontium, barium, radium, or any combination thereof. The metal carbonyl additive may be a salt. The metal carbonyl additive may be a silicon salt in the form of R₃Si—, where R is independently selected from a phenyl, halophenyl, hydrogen, alkyl, alkylhalo, alkoxy, or any combination thereof. The metal carbonyl additive may be NaCo(CO)₄, Co₂(CO)₈, HCo(CO)₄, or any combination thereof. Where a metal centered compound containing a halogen is formed after the metalation step, NaCo(CO)₄ may be used to form the carbonylation catalyst. Where a Metal centered compounds containing an alkyl group is formed, Co₂(CO)₈ or HCo(CO)₄ may be used to form the carbonylation catalyst.

In some metal centered compounds, one or more polar ligands may coordinate to M, or a combination thereof and fill the coordination valence of the metal atom. The polar ligand may be a solvent. The polar ligand may be any compound with at least two free valence electrons. The polar ligand may be aprotic. The compound may be tetrahydrofuran, dioxane, diethyl ether, acetonitrile, carbon disulfide, pyridine, epoxide, ester, lactone, or a combination thereof.

The solvent may be a polar aprotic solvent, a polar protic solvent, or a nonpolar solvent that functions to dissolve one or more compounds described herein. One solvent may be soluble in one or more other solvents to increase solubility of one or more of the compounds described herein. A first solvent may be combined with a second solvent that is miscible in the first solvent to precipitate components that are insoluble in the second solvent. The solvents may be selected to form an organic phase or an aqueous phase layer that is distinct from another aqueous phase layer, another organic phase layer, a precipitate, or any some combination. The solvent may be one or more of water, methanol, ethanol, propanol, hexane, heptane, nonane, decane, tetrahydrofuran, methyltetrahydrofuran, diethyl ether, sulfolane, toluene, pyridine, diethyl ether, 1,4-dioxane, acetonitrile, ethyl acetate, dimethoxy ethane, acetone, chloroform, dichloromethane, or any combination thereof.

Several techniques have been theorized to illustrate the teaching of the present disclosure. Each teaching is simply an example of the disclosure and is not intended to limit the teachings to any single technique.

FIG. 1 is a synthetic scheme to form a carbonylation catalyst. To a round bottom flask, 70.0 mmol of ketone is mixed with 140 mL of dichloromethane. The reaction mixture is cooled to 0° C. and 105 mmol of alkyl or aryl magnesium bromide is added to the round bottom flask over a 10 minute period. The solution is then allowed to warm to room temperature and is stirred overnight. The reaction is cooled to 0° C. and is quenched with a saturated ammonium chloride solution. The organic and aqueous layers are separated, and the organic layer is collected which is dried with sodium sulfate or magnesium sulfate. The solvent is removed under reduced pressure and a tertiary alcohol is purified by column chromatography on silica gel. The tertiary alcohol (83.4 mmol) is dissolved in 90 mL of dichloromethane and is cooled to 0° C. in a round bottom flask equipped with a magnetic stir bar under ambient air. To the tertiary alcohol dissolved in dichloromethane, 250 mmol of triethylamine is added and stirred for 1 minute, and 167 mmol of methanesulfonic anhydride that is dissolved in 30 ml of dichloromethane and is added slowly over a 5 minute period. The reaction is stirred at 0° C. for 60 minutes. Then brine and water are added to the reaction mixture and the organic layer is separated from the aqueous phase and is concentrated by reduced pressure. The product alkene is purified by dissolving in hexanes and passing the alkene over a frit with a layer of silica gel. To a solution of 40.0 mmol of 4-substituted phenol and 60.0 mmol of the product alkene in 20 ml of dichloromethane, 30.0 methane sulfonic acid is added dropwise. The solution is allowed to stir overnight at room temperature. Water and dichloromethane are added, and the layers are separated. The organic layer is washed with saturated sodium bicarbonate and dried with sodium sulfate or magnesium sulfate. The product 2,4-disubstutted phenol is purified by column chromatography over silica gel. Under an atmosphere of nitrogen is added 30.0 mmol of 2,4-disubstituted phenol, 45.0 mmol of 2,6-lutidine in 100 ml of toluene. The solution is reduced in temperature to 0° C. and 15 mmol of tin (IV) chloride is added slowly through a septum. The solution is allowed to stir at room temperature for 20 minutes and then 150 mmol of paraformaldehyde is added. The flask is put under a slight vacuum and heated 90° C. overnight. After cooling to room temperature, 2 M HCl is added and stirred for 1 hour. Diethyl ether is added, and the layers are separated. The organic layer is washed with sodium bicarbonate and brine, and subsequently the organic layer is dried with sodium sulfate or magnesium sulfate. The solvent is removed by reduced pressure to yield the desired product.

The ligand synthesis is completed by reacting the salicylaldehyde with orthophenylene diamine in a ratio of 2.2 to 1. Salicylaldehyde in an amount of 0.004 mol is dissolved in 30 mL of ethanol (EtOH) and added 0.00176 mol ortho-phenylene diamine and refluxed overnight. The temperature subsequently is reduced and the product is isolated by gravity filtration after precipitation of the ligand from the EtOH by using standard methods for condensation reaction between aldehyde of the salicylaldehyde and a diamine. After the ligand is formed, a reaction vessel under an inert atmosphere is charged with 0.1 mol of ligand in 100 mL toluene solvent (1.0 M). One equivalent of Et₂AlCl is added to the solution slowly and allowed to stir at room temperature overnight. Product is collected by filtration after precipitation from solution. To form the carbonylation catalyst, a reaction vessel under an inert atmosphere is charged with 0.1 mol of metalated ligand in 100 mL THF solvent (1.0 M). One equivalent of NaCo(CO)₄ is added to the solution slowly and allowed to stir at room temperature overnight. Product is filtered to removed NaCl byproduct. The product is collected by precipitating the catalyst from THE solvent by addition of anti-solvent hexanes which is then collected by filtration.

### Enumerated Embodiments

The following examples are provided to illustrate the invention but are not intended to limit the scope thereof. All parts and percentages are by weight unless otherwise indicated.

Embodiment 1. A composition, comprising:
a metal carbonyl anion; and
a cation ionically bonded to the metal carbonyl anion, comprising:
a ligand including two residues of 3,5-substituted salicylaldehydes connected by an hydrocarbyl-diimine bridge that includes a nitrogen atom contacted with a carbon of an aldehyde residue at each of the two residues of the 3,5-substituted salicylaldehydes, each of the residues of the 3,5-substituted salicylaldehydes independently substituted at one or both of a 3 position and a 5 position by a hydrocarbyl group containing at least 5 carbons;
a metal coordinated with the ligand at each hydroxyl residue the two residues of the 3,5-substituted salicylaldehydes at a 2 position and at each of the nitrogen atoms of the hydrocarbyl-diimine bridge; and
two polar ligands coordinated with the metal

Embodiment 2. The composition of embodiment 1, wherein the composition, comprises:

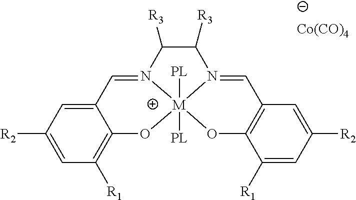

wherein M is a metal;
wherein PL is a polar ligand;
wherein each R₁ is independently selected from one or more of an alkyl group, an aryl group, or an alkyl-aryl group;
wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
wherein at least one of R₁ or R₂ is selected from an alkyl group, an aryl group, or an alkyl-aryl group that contains at least 5 carbons; and
wherein each R₃ is independently selected from hydrogen, methyl, a C₂₋₁₀ alkyl group, a combination thereof, or in combination form a six membered ring that is optionally a substituted aromatic ring

Embodiment 3. The composition of embodiment 2, wherein each R₁ is independently selected from one or more of a 1,1 ethylphenyl propane, 2,2 methylphenyl ethane, 1,2-trimethyl propyl, 1,1-dimethylpropyl, 1-methylcyclohexyl, 1,1-diphenylethyl, or any combination thereof.

Embodiment 4. The composition of embodiments 2 or 3, wherein each R₂ is independently selected from one or more of a methyl group, a C₂₋₁₆ alkyl group, an aryl group, an alkyl-aryl group, or any combination thereof.

Embodiment 5. The composition of embodiment 1, wherein each of the 3,5-substituted phenyls are independently substituted at the 3 position by one or more of 1,1 ethylphenyl propane, 2,2 methylphenyl ethane, 1,2-trimethyl propyl, 1,1-dimethylpropyl, 1-methylcyclohexyl, 1,1-diphenylethyl, or any combination thereof.

Embodiment 6. The composition of any one of embodiments 1-5, wherein the composition comprises one or more of the following compounds:

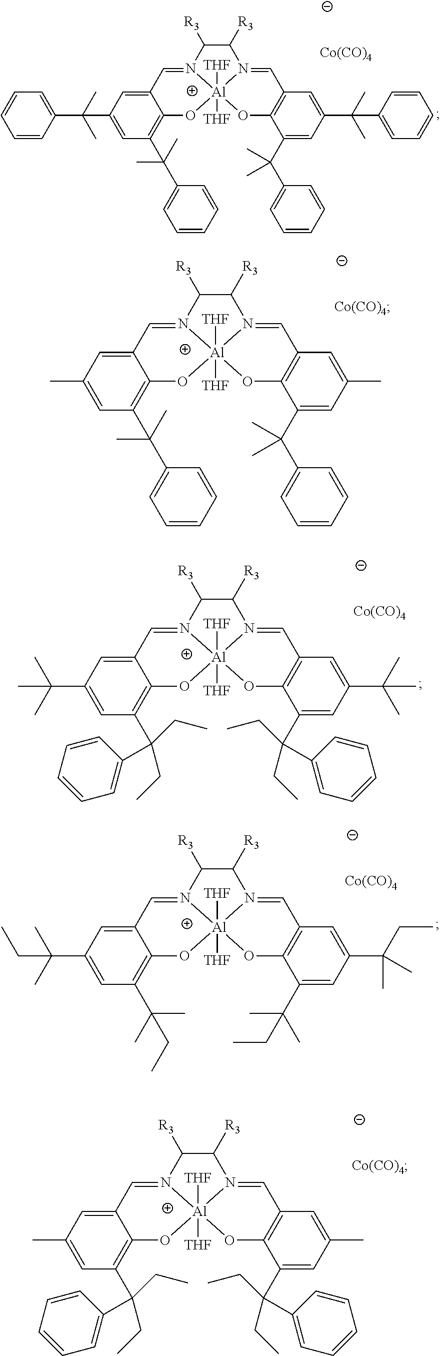

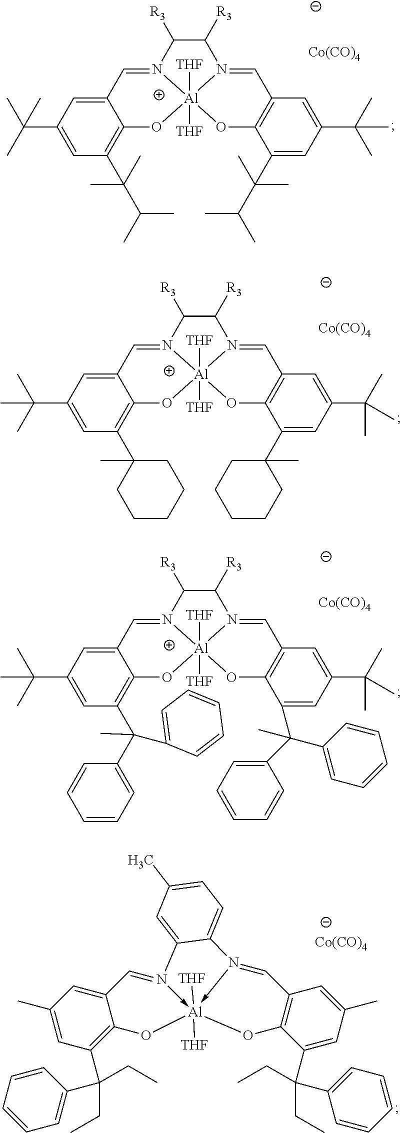

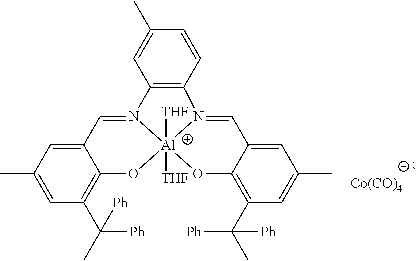

or any combination thereof,
   wherein each R₃ is independently selected from hydrogen, methyl, in combination form one or more cyclohexane, cyclohexene, or aromatic rings that are optionally substituted, or any combination thereof.

Embodiment 7. The composition of any one of the preceding embodiments, wherein the ligand, comprises:

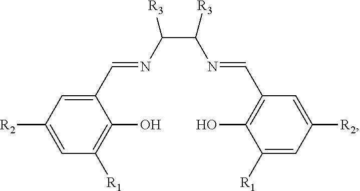

wherein each R₁ is independently selected from one or more of an alkyl group, an aryl group, or an alkyl-aryl group;
wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
wherein at least one of R₁ or R₂ is selected from an alkyl group, an aryl group, or an alkyl-aryl group that contains at least 5 carbons; and
wherein each R₃ is independently selected from hydrogen, methyl, a C₂₋₁₀ alkyl group, a combination thereof, or in combination form a six membered ring that is optionally a substituted aromatic ring. Embodiment 8. The composition of any one of the preceding embodiments, wherein the cation includes a salph, a salen, a salpn, salcy, or salalen ligands.

Embodiment 9. The composition of any one of the preceding embodiments, wherein the metal carbonyl anion comprises a metal and one or more carbonyls contacting the metal.

Embodiment 10. The composition of any one of the preceding embodiments, wherein the metal carbonyl anion comprises (Co(CO)₄).

Embodiment 11. The composition of any one of the preceding embodiments, wherein the hydrocarbyl-diimine bridge forms a portion of one or more cyclohexane, a cyclohexene, or aromatic rings that may be optionally substituted.

Embodiment 12. The composition of any one of the preceding embodiments, wherein the cyclohexane, cyclohexene, aromatic rings is optionally substituted by one or more methyl groups, a C₂₋₁₀ alkyl group, or any combination thereof.

Embodiment 13. The composition of any one of the preceding embodiments, wherein the hydrocarbyl-diimine bridge comprises an ethyldiimine bridge that is substituted at a 1 position, a 2 position, or both by one or more of a hydrogen group, a methyl group, a C₂₋₁₀ alkyl group, or any combination thereof.

Embodiment 14. The composition of any one of the preceding embodiments, wherein the metal includes one or more of Al or Cr.

Embodiment 15. The composition of any one of the preceding embodiments, wherein the R₂ group is independently selected from one or more of hydrogen, methyl, a —C₂-C₁₂ alkyl group, or any combination thereof.

Embodiment 16. The composition of any one of the preceding embodiments, wherein the polar ligand includes one or more of tetrahydrofuran, dioxane, diethyl ether, or any combination thereof.

Embodiment 17. The composition of any one of the preceding embodiments, wherein the composition has catalytic activity with one or more epoxides to form one or more lactones.

Embodiment 18. The composition of any one of the preceding embodiments, wherein the ligand has an aromatic ring at one or more of the 3 positions, the 5 positions, or any combination thereof.

Embodiment 19. The composition of any one of the preceding embodiments, wherein the ligand has aromaticity at one or more of the 3 positions, the 5 positions, or any combination thereof.

Embodiment 20. A method, comprising:
contacting a ligand with a metalating agent to form a metal centered compound, wherein the ligand includes the residues of two 3, 5-substituted salicylaldehydes connected by an hydrocarbyl-diimine bridge at the aldehyde residue of the two residues of the 3,5-substituted salicylaldehydes, wherein each substitution at one or both of a 3 position and a 5 position comprises a hydrocarbyl group containing at least 5 carbons; and
contacting the metal centered compound with a metal carbonyl and a polar ligand to form a composition.

Embodiment 21. The method of embodiment 20, wherein the method comprises:
contacting a compound according to the following formula:

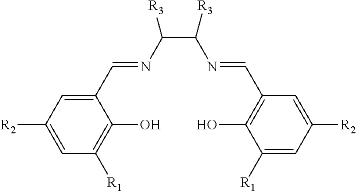

with a metalating agent to form one of the compounds according to the following formula:

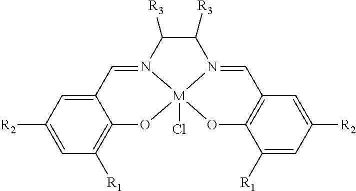

contacting the formed compounds with a metal carbonyl and a polar ligand under conditions so that one of the following compositions are formed:

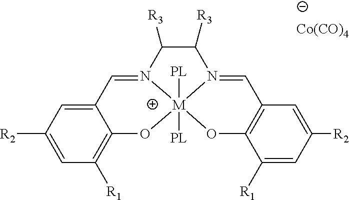

wherein M is a metal;
wherein each PL is the polar ligand;
wherein each R₁ is independently selected from one or more of an alkyl group, an aryl group, or an alkyl-aryl group;
wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
wherein at least one of R₁ or R₂ is selected from an alkyl group, an aryl group, or an alkyl-aryl group that contains at least 5 carbons; and
wherein each R₃ is independently selected from hydrogen, methyl, a C₂₋₁₀ alkyl group, a combination thereof, or in combination form a six membered ring that is optionally a substituted aromatic ring.

Embodiment 22. The method of embodiment 21, wherein each R₁ is independently selected from one or more of 1,1 ethylphenyl propane, 2,2 methylphenyl ethane, 1,2-trimethyl propyl, 1,1-dimethylpropyl, 1-methylcyclohexyl, 1,1-diphenylethyl, or any combination thereof.

Embodiment 23. The method of embodiments 21 or 22, wherein each R₂ is independently selected from one or more of methyl group, a C₁₋₁₆ alkyl group, an aryl group, an alkyl-aryl group, or any combination thereof.

Embodiment 24. The method of embodiments 20-23, further comprising preparing the 3, 5-substituted salicylaldehydes connected by an hydrocarbyl-diimine bridge or the compound according to the following formula:

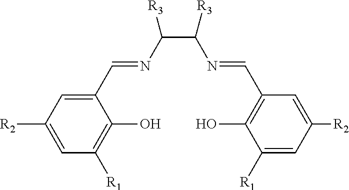

by:
contacting a carbonyl di-substituted at the carbon with a hydrogen, a —C₁-C₁₂ alkyl group, or an aryl group with a Grignard reagent under such conditions so that a —C₁-C₁₂ alkyl group or an aryl group is added to the carbonyl and the oxygen atom converts to a tertiary alcohol to form a trisubstituted tertiary alcohol;
contacting the trisubstituted tertiary alcohol with methane sulfonic anhydride under such conditions so that an alkene is formed which is 1,1-disubstituted with two or more of a hydrogen, a —C₁-C₁₂ alkyl group, or an aryl group;
contacting a phenol which is optionally substituted at the 4 position with a hydrogen, methyl, a —C₂-C₁₂ alkyl group, an aryl group, a t-butyl alkyl group, a halogen, an amine, —CF₃, hydrocarbyl oxy group, or —NO₂ with the alkene which is 1,1-disubstituted with two or more of a hydrogen, a —C₁-C₁₂ alkyl group, or an aryl group in the presence of an acid catalyst under such conditions so that the alkene is added at the 2 position of the aromatic ring to form a phenol that is substituted at the 2 position with the alkene and which is optionally substituted at the 4 position;
contacting the 2-alkene phenol which is optionally substituted at the 4 position with a formaldehyde or a formaldehyde precursor in the presence of a Lewis acid catalyst and a base under such conditions so that a salicylaldehyde is formed which is substituted at the 3 position by the alkene and is optionally substituted at the 5 position by a hydrogen, methyl, a —C₂-C₁₂ alkyl group, an aryl group, a t-butyl alkyl group, a halogen, an amine, —CF₃, hydrocarbyl oxy group, or —NO₂; and
contacting the 3,5-substituted salicylaldehyde with a diamine under such conditions so that the 3, 5-substituted salicylaldehydes connected by an hydrocarbyl-diimine bridge or the compound according to the formula above is formed.

Embodiment 25. The method of any one of embodiments 20-24, wherein the polar ligand includes one or more of tetrahydrofuran, diethyl ether, dioxane, or any combination thereof.

Embodiment 26. The method of any one of embodiments 20-25, wherein the R₂ is independently selected from one or more of hydrogen, —CH₃, a —C₂-C₁₂ alkyl group, or an aryl group.

Embodiment 27. The method of any one of embodiments 20-26, wherein the metal includes one or more of Al or Cr.

Embodiment 28. The method of any one of embodiments 20-27, wherein the metalating agent includes one or more of (Et)₂AlCl, (Et)₃Al, CrCl₂, or any combination thereof.

Embodiment 29. The method of any one of embodiments 20-28, wherein the diamine includes one or more of ethylene diamine, ortho-phenylene diamine, ortho-cyclohexyl diamine, phenanthroline, bipyridine, substituted ethylene diamine, substituted phenylene diamine, substituted ortho-cyclohexyl diamine, substituted phenanthroline, or substituted bipyridine.

Embodiment 30. The method of any one of embodiments 20-29, wherein the metal carbonyl includes one or more of NaCo(CO)₄, Co₂(CO)₈, HCo(CO)₄, or a combination thereof.

Embodiment 31. The method of any one of embodiments 20-30, wherein the acid catalyst includes one or more of methane sulfonic acid.

Embodiment 32. The method of any one of embodiments 20-31, wherein the Lewis acid catalyst includes SnCl₄, AlCl₃, FeCl₃, or any combination thereof.

Embodiment 33. The method of any one of embodiments 20-32, wherein the Grignard reagent includes one or more of alkyl magnesium bromide, aryl magnesium bromide.

Embodiment 34. The method of any one of embodiments 20-33, wherein the base is 2,6-lutidine, triethylamine, or both.

Embodiment 35. The method of any one of embodiments 20-35, wherein the formaldehyde or formaldehyde precursor is paraformaldehyde.

Embodiment 36. A method, comprising contacting an epoxide and carbon monoxide in the presence of a carbonylation catalyst to form beta propiolactone,
wherein the carbonylation catalyst comprises:
   a metal carbonyl anion; and
   a cation ionically bonded to the metal carbonyl anion, comprising:
      a ligand including two residues of 3,5-substituted salicylaldehydes connected by an hydrocarbyl-diimine bridge that includes a nitrogen atom contacted with a carbon of an aldehyde residue at each of the two residues of the 3,5-substituted salicylaldehydes, each of the residues of the 3,5-substituted salicylaldehydes independently substituted at one or both of a 3 position and a 5 position by a hydrocarbyl group containing at least 5 carbons; and
      a metal coordinated with the ligand at each hydroxyl residue of a 2 position of the two residues of the 3,5-substituted salicylaldehydes and at each of the nitrogen atoms of the hydrocarbyl-diimine bridge; and
      two polar ligands coordinated with the metal.

Embodiment 37. The method of embodiment 36, wherein the carbonylation catalyst has a structure according to the following formula:

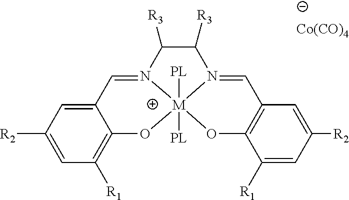

wherein M is a metal;
wherein PL is a polar ligand;
wherein each R₁ is independently selected from one or more of an alkyl group, an aryl group, or an alkyl-aryl group;
wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
wherein at least one of R₁ or R₂ is selected from an alkyl group, an aryl group, or an alkyl-aryl group that contains at least 5 carbons; and
wherein each R₃ is independently selected from hydrogen, methyl, a C₂₋₁₀ alkyl group, a combination thereof, or in combination form a six membered ring that is optionally a substituted aromatic ring.

Embodiment 38. The method of embodiments 36 or 37, wherein the polar ligand is tetrahydrofuran, dioxane, diethyl ether, or any combination thereof.

Embodiment 39. The method of any one of embodiments 36-38, wherein the metal is Al or Cr.

Embodiment 40. The method of any one of embodiments 36-39, wherein R₂ is independently selected from one or more of H, —CH₃, a —C₂-C₁₂ alkyl group, or an aryl group.

Embodiment 41. The method of any one of embodiments 36-40, wherein R₁ is independently selected from one or more of 1,1 ethylphenyl propane, 2,2 methylphenyl ethane, 1,2-trimethyl propyl, 1,1-dimethylpropyl, 1-methylcyclohexyl, 1,1-diphenylethyl, or any combination thereof.

### Examples

The following examples are provided to illustrate the disclosure but are not intended to limit the scope thereof.

The NMR analysis is conducted on a Varian Mercury spectrometer operating at 300.1 MHz. The sample is dissolved in THF-d8 before testing.

The FTIR analysis is conducted on a Nicolet iS5 equipped with an iD1 Transmission accessory to characterize each of the disclosed examples. The sample is dissolved in THF before testing.

The in-situ-FTIR analysis tracking the catalytic activity of the carbonylation catalyst is conducted on a Mettler Toledo ReatIR 45 m equipped with a silicone tipped sentinel that is directly affixed to the bottom of the reactor.

TABLE 1
Data Corresponding to Conversion of Ethylene Oxide

| | | | Bridging | dbPL/dt | dbPL/dt |
|---|---|---|---|---|---|
| | | | Ligand | (at 10% EO | (at 25% EO |
| Catalyst | R₁ | R₂ | Precursor | conversion) | conversion) |
| 1 | C((CH₃)₃) | C((CH₃)₃) | OPD | 7.5 | 0.6 |
| 2 | C((CH₃)₂(C₆H₅) | C((CH₃)₂(C₆H₅) | OPD | 43.4 | 34.3 |
| 3 | C((CH₃)₂(C₆H₅) | CH₃ | OPD | 46.8 | 39.0 |
| 4 | C((C₂H₅)₂(C₆H₅) | C((CH₃)₃) | OPD | 47.7 | 44.4 |
| 5 | C((C₂H₅)₂(C₆H₅) | CH₃ | OPD | 61.8 | 63.6 |
| 6 | C(CH₃)₂CH(CH₃)₂ | C((CH₃)₃) | OPD | 8.1 | 6.8 |
| 7 | C(CH₃)₂(C₂H₅) | C(CH₃)₂(C₂H₅) | OPD | 10.6 | 2.9 |
| 8 | C(CH₃)(C₆H₁₁)H | C((CH₃)₃) | OPD | 0.5 | 0 |
| 9 | C(C₆H₅)₂(CH₃) | CH₃ | OPD | 41.6 | 31.2 |
| 10 | C((C₂H₅)₂(C₆H₅) | CH₃ | 4-CH₃— | 73.6 | 83.4 |
| | | | OPD | | |
| 11 | C(C₆H₅)₂(CH₃) | CH₃ | 4-CH₃— | 47.9 | 54.4 |
| | | | OPD | | |

OPD = o-phenylene diamine; 4-CH₃—OPD = 4-methyl-o-phenylene diamine

In the above table 1, the R₁ and R₂ groups correspond to the above Example Component I to illustrate Examples 1-9. To test each Example, carbon monoxide and ethylene oxide is reacted in the presence of the Example catalyst in a reactor at a temperature of 70 degrees Celsius and 900 psi. The carbon monoxide is added in excess, and the ethylene oxide and the Example catalyst are added in a molar ratio of 800:1 such that beta propiolactone is formed. Each Example catalyst is added in an amount of 0.06 mmol. The Example catalyst, the ethylene oxide, and the carbon monoxide are reacted in the presence of 70 ml of tetrahydrofuran.

The conversion rates above are measured as the rate of beta propiolactone formation per minute (dBPL/dt) that is normalized by catalyst concentration. Conversion rates of ethylene oxide to beta propiolactone are tabulated at 10% and 25% ethylene oxide conversion points.

As shown above and in FIGS. 2 and 3, compositional variations to the tetradentate Schiff base ligand yield carbonylation catalysts with higher activity as compared to the incumbent catalyst comprised Schiff base with an Al Lewis acid metal center, Example 1. Steric and/or electronic effects related to added aromaticity at the 3,3′ positions show benefits for enhancing catalytic activity, which is highlighted in Table 1. It is understood that the rate determining step (RDS) in the carbonylative ring expansion of epoxides is ring closure to form the lactone. The imparted steric/electronic variations by the combination of the alkyl and/or aromatic groups in Examples 2-11 are thus assisting in facilitating the ring closure increasing the rate of beta propiolactone formation.

Regarding the characterization data, the following confirm each of the examples below through H NMR and FTIR analysis.

FIG. 4 is a 1H NMR spectrum of the isolated carbonylation catalyst, Example 1, analyzed in d8-THF solvent.

FIG. 5 is a FTIR spectrum of the isolated carbonylation catalyst, Example 1, depicting the carbonyl stretching peak associated with Co(CO)₄⁻ located at 1886 cm⁻¹ analyzed in THF solvent.

FIG. 6 is a 1H NMR spectrum of the isolated carbonylation catalyst, Example 2, analyzed in d8-THF solvent.

FIG. 7 is a FTIR spectrum of the isolated carbonylation catalyst, Example 2, depicting the carbonyl stretching peak associated with Co(CO)₄⁻ located at 1886 cm⁻¹ analyzed in THF solvent.

FIG. 8 is a 1H NMR spectrum of the isolated carbonylation catalyst, Example 3, analyzed in d8-THF solvent.

FIG. 9 is a FTIR spectrum of the isolated carbonylation catalyst, Example 3, depicting the carbonyl stretching peak associated with Co(CO)₄⁻ located at 1886 cm⁻¹ analyzed in THF solvent.

FIG. 10 is a 1H NMR spectrum of the isolated carbonylation catalyst, Example 4, analyzed in d8-THF solvent.

FIG. 11 is a FTIR spectrum of the isolated carbonylation catalyst, Example 4, depicting the carbonyl stretching peak associated with Co(CO)₄⁻ located at 1886 cm⁻¹ analyzed in THF solvent.

FIG. 12 is a 1H NMR spectrum of the isolated carbonylation catalyst, Example 5, analyzed in d8-THF solvent.

FIG. 13 is a FTIR spectrum of the isolated carbonylation catalyst, Example 5, depicting the carbonyl stretching peak associated with Co(CO)₄⁻ located at 1886 cm⁻¹ analyzed in THE solvent.

FIG. 14 is a 1H NMR spectrum of the isolated carbonylation catalyst, Example 6, analyzed in d8-THF solvent.

FIG. 15 is a FTIR spectrum of the isolated carbonylation catalyst, Example 6, depicting the carbonyl stretching peak associated with Co(CO)₄⁻ located at 1886 cm⁻¹ analyzed in THF solvent.

FIG. 16 is a 1H NMR spectrum of the isolated carbonylation catalyst, Example 7, analyzed in d8-THF solvent.

FIG. 17 is a FTIR spectrum of the isolated carbonylation catalyst, Example 7, depicting the carbonyl stretching peak associated with Co(CO)₄⁻ located at 1886 cm⁻¹ analyzed in THF solvent.

FIG. 18 is a 1H NMR spectrum of the isolated carbonylation catalyst, Example 8, analyzed in d8-THF solvent.

FIG. 19 is a FTIR spectrum of the isolated carbonylation catalyst, Example 8, depicting the carbonyl stretching peak associated with Co(CO)₄⁻ located at 1886 cm⁻¹ analyzed in THF solvent.

FIG. 20 is a 1H NMR spectrum of the isolated carbonylation catalyst, Example 9, analyzed in d8-THF solvent.

FIG. 21 is a FTIR spectrum of the isolated carbonylation catalyst, Example 9, depicting the carbonyl stretching peak associated with Co(CO)₄⁻ located at 1886 cm⁻¹ analyzed in THF solvent.

FIG. 22 is a 1H NMR spectrum of the isolated carbonylation catalyst, Example 10, analyzed in d8-THF solvent.

FIG. 23 is a FTIR spectrum of the isolated carbonylation catalyst, Example 10, depicting the carbonyl stretching peak associated with Co(CO)₄⁻ located at 1886 cm-1 analyzed in THF solvent.

FIG. 24 is a 1H NMR spectrum of the isolated carbonylation catalyst, Example 11, analyzed in d8-THF solvent.

FIG. 25 is a FTIR spectrum of the isolated carbonylation catalyst, Example 11, depicting the carbonyl stretching peak associated with Co(CO)₄⁻ located at 1887 cm⁻¹ analyzed in THF solvent.

Interpretation of the 1H NMR and FTIR spectra collected for the Example catalysts show that the desired material is successfully synthesized and isolated.

The invention claimed is:

1. A composition, comprising:
a) a metal carbonyl anion; and
b) a cation ionically bonded to the metal carbonyl anion, comprising:
i) a ligand including two residues of 3,5-substituted salicylaldehydes connected by a hydrocarbyl-diimine bridge that includes a nitrogen atom contacted with a carbon of an aldehyde residue at each of the two residues of the 3,5-substituted salicylaldehydes, wherein each of the residues of the 3,5-substituted salicylaldehydes are independently substituted at 3 position by a hydrocarbyl group that is a branched alkyl-aryl group, and wherein each of the residues of the 3,5-substituted salicylaldehydes are independently substituted at a 5 position by a hydrogen, methyl, a —C₂-C₁₂, alkyl group, an aryl group, a t-butyl alkyl group, a halogen, an amine, —CF₅, hydrocarbyl oxy group, or —NO₂;
ii) a metal coordinated with the ligand at each hydroxyl residue the two residues of the 3,5-substituted salicylaldehydes at a 2 position and at each of the nitrogen atoms of the hydrocarbyl-diimine bridge; and
iii) two polar ligands coordinated with the metal.

2. The composition of claim 1, wherein the composition, comprises: 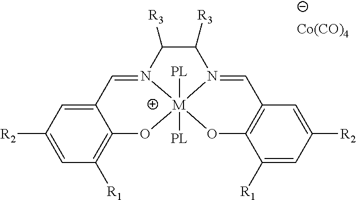
wherein M is a metal;
wherein PL is a polar ligand;
wherein each R₁ is independently a hydrocarbyl group comprising a branched alky-aryl group;
wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
wherein at least one of R₁ or R₂ is selected from an alkyl group, an aryl group, or an alkyl-aryl group that contains at least 5 carbons; and
wherein each R₃ is independently selected from hydrogen, methyl, a C₂₋₁₀ alkyl group, a combination thereof, or in combination form a six membered ring that is optionally a substituted aromatic ring.

3. The composition of claim 2, wherein each R₁ is independently selected from one or more of a 1,1 ethylphenyl propane, 2,2 methylphenyl ethane, 1,1-diphenylethyl, or any combination thereof.

4. The composition of claim 2, wherein each R₂ is independently selected from one or more of a methyl group, a C₂₋₁₆ alkyl group, an aryl group, an alkyl-aryl group, or any combination thereof.

5. The composition of claim 1, wherein each of the 3,5-substituted phenyls are independently substituted at the 3 position by one or more of 1,1 ethylphenyl propane, 2,2 methylphenyl ethane, 1,1-diphenylethyl, or any combination thereof.

6. The composition of claim 1, wherein the ligand, comprises: 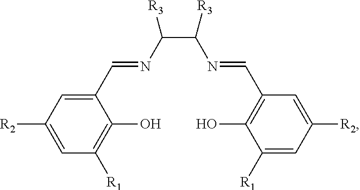
wherein each R₁ is independently a hydrocarbyl group comprising a branched alkyl-aryl group;
wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
wherein at least one of R₁ or R₂ is selected from an alkyl group, an aryl group, or an alkyl-aryl group that contains at least 5 carbons; and
wherein each R₃ is independently selected from hydrogen, methyl, a C₂₋₁₀ alkyl group, a combination thereof, or in combination form a six membered ring that is optionally a substituted aromatic ring.

7. (canceled)

8. (canceled)

9. The composition claim 2, wherein the metal carbonyl anion comprises (Co(CO)₄).

10. (canceled)

11. (canceled)

12. (canceled)

13. The composition of claim 2, wherein the metal includes one or more of Al or Cr.

14. (canceled)

15. (canceled)

16. (canceled)

17. (canceled)

18. A method, comprising:
a) contacting a ligand with a metalating agent to form a metal centered compound, wherein the ligand includes the residues of two 3, 5-substituted salicylaldehydes connected by an hydrocarbyl-diimine bridge at the aldehyde residue of the two residues of the 3,5-substituted salicylaldehydes, wherein each substitution at a 3 position comprises a hydrocarbyl group comprising an alkyl-aryl group and wherein each substitution of the 3,5-substituted salicylaldehydes at a 5 position comprises hydrogen, methyl, a —C₂-C₁₂, alkyl group, an aryl group, a t-butyl alkyl group, a halogen, an amine, —CF₅, hydrocarbyl oxy group, or —NO₂; and
b) contacting the metal centered compound with a metal carbonyl and a polar ligand to form a composition.

19. The method of claim 18, wherein the method comprises:
a) contacting a compound according to the following formula: 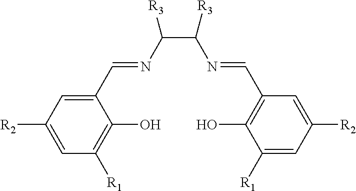
with a metalating agent to form one of the compounds according to the following formula: 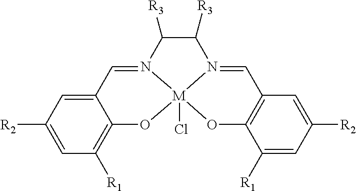
b) contacting the formed compounds with a metal carbonyl and a polar ligand under conditions so that one of the following compositions are formed: 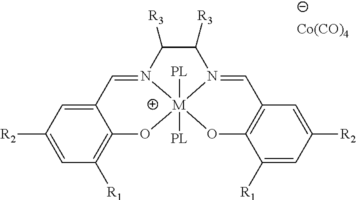
wherein M is a metal;
wherein each PL is the polar ligand;
wherein each R₁ is independently a hydrocarbyl group comprising a branched alkyl-aryl group;
wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
wherein at least one of R₁ or R₂ is selected from an alkyl group, an aryl group, or an alkyl-aryl group that contains at least 5 carbons; and
wherein each R₃ is independently selected from hydrogen, methyl, a C₂₋₁₀ alkyl group, a combination thereof, or in combination form a six membered ring that is optionally a substituted aromatic ring.

20. The method of claim 19, wherein each R₁ is independently selected from one or more of 1,1 ethylphenyl propane, 2,2 methylphenyl ethane, 1,1-diphenylethyl, or any combination thereof.

21. The method of claim 19, wherein each R₂ is independently selected from one or more of methyl group, a C₁₋₁₆ alkyl group, an aryl group, an alkyl-aryl group, or any combination thereof.

22. The method of claim 19, wherein the polar ligand includes one or more of tetrahydrofuran, diethyl ether, dioxane, or any combination thereof.

23. The method of claim 19, wherein the R₂ is independently selected from one or more of hydrogen, —CH₃, a —C₂-C₁₂ alkyl group, or an aryl group.

24. The method of any one of claim 19, wherein the metal includes one or more of Al or Cr.

25. The method of claim 19, wherein the metalating agent includes one or more of (Et) 2AlCl, (Et) 3Al, CrCl₂, or any combination thereof, and/or wherein the metal carbonyl includes one or more of NaCo(CO)₄, Co₂ (CO) 8, HCo(CO)₄, or a combination thereof.

26. (canceled)

27. A method, comprising contacting an epoxide and carbon monoxide in the presence of a carbonylation catalyst to form beta propiolactone,
wherein the carbonylation catalyst comprises:
c) a metal carbonyl anion; and
d) a cation ionically bonded to the metal carbonyl anion, comprising:
i) a ligand including two residues of 3,5-substituted salicylaldehydes connected by a hydrocarbyl-diimine bridge that includes a nitrogen atom contacted with a carbon of an aldehyde residue at each of the two residues of the 3,5-substituted salicylaldehydes, each of the residues of the 3,5-substituted salicylaldehydes independently substituted at one or both of a 3 position and a 5 position by a hydrocarbyl group comprising an alkyl-aryl group containing at least 5 carbons;
ii) a metal coordinated with the ligand at each hydroxyl residue of a 2 position of the two residues of the 3,5-substituted salicylaldehydes and at each of the nitrogen atoms of the hydrocarbyl-diimine bridge; and
iii) two polar ligands coordinated with the metal.

28. The method of claim 27, wherein the carbonylation catalyst has a structure according to the following formula: 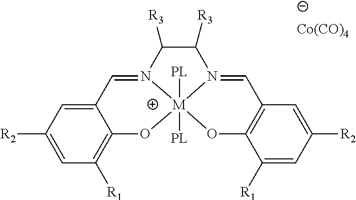
wherein M is a metal;
wherein PL is a polar ligand;
wherein each R₁ is independently hydrocarbyl group comprising a branched alkyl-aryl group;
wherein each R₂ is independently selected from one or more of a hydrogen atom, a methyl group, a C₂₋₁₆ alkyl group, an aryl group, an alkyl-aryl group, a halogen, an amine, a trifluoromethyl group, a nitro group, a hydrocarbyl oxy group, or any combination thereof;
wherein at least one of R₁ or R₂ is selected from an alkyl group, an aryl group, or an alkyl-aryl group that contains at least 5 carbons; and
wherein each R₃ is independently selected from hydrogen, methyl, a C₂₋₁₀ alkyl group, a combination thereof, or in combination form a six membered ring that is optionally a substituted aromatic ring.

29. (canceled)

30. The method of claim 27, wherein the metal is Al or Cr.

31. The method of claim 27, wherein R₁ is independently selected from one or more of 1,1 ethylphenyl propane, 2,2 methylphenyl ethane, 1,1-diphenylethyl, or any combination thereof.
